(19) United States
(12) Patent Application Publication
(10) Pub. No.: US 2023/0075190 A1
(43) Pub. Date: Mar. 9, 2023

(54) COMPOSITIONS AND METHODS OF USE THEREOF FOR TREATMENT OR PREVENTION OF CONDITIONS ASSOCIATED WITH HYPERPROLIFERATIVE CELLULAR DIVISION, DIABETES, IMMUNODEFICIENCY DISEASES, ALZHEIMER'S DISEASE OR AUTOIMMUNE DISEASES, AND TESTING SYSTEM AND METHOD

(71) Applicant: Chai Foundation for Medical Research & Life Extension, Inc., Phoenix, AZ (US)

(72) Inventors: Eric R. Brown, Des Plaines, IL (US); Eroca Daniel, Phoenix, AZ (US)

(21) Appl. No.: 17/896,847

(22) Filed: Aug. 26, 2022

Related U.S. Application Data
(60) Provisional application No. 63/239,714, filed on Sep. 1, 2021.

(51) Int. Cl. A61K 39/00; A61K 39/05; A61P 35/00; A61P 3/10; A61P 37/00; A61P 25/28; G01N 33/50

(52) U.S. Cl. CPC A61K 39/0002; A61K 39/05; A61P 35/00; A61P 3/10; A61P 37/00; A61P 25/28; G01N 33/5088; A61K 2039/585; A61K 2039/58

(57) ABSTRACT

The present invention is directed to compositions, kits, and methods for the treatment of conditions associated with hyperproliferative cellular division, such as cancer, diabetes, pre-diabetes, conditions associated with diabetes or pre-diabetes, immune deficiency disease or disorder, conditions associated with the immune deficiency disease or disorder, Alzheimer's disease and conditions associated with Alzheimer's disease, and/or an autoimmune disease and conditions associated with autoimmune diseases. One aspect provides a composition extracted from a biological fluid, such as plasma, from an animal inoculated with an antigenic agent, such as an Ascomyceta (e.g., Aspergillus). One aspect provides for a method of producing such composition. One aspect provides a method for treating conditions associated with hyperproliferative cellular division, diabetes, pre-diabetes, conditions associated with diabetes or pre-diabetes, immune deficiency disease or disorder, or conditions associated with immune deficiency disease or disorder, Alzheimer's disease or conditions associated with Alzheimer's disease, or an autoimmune disease or conditions associated with autoimmune diseases, by administering such a composition to a subject in need thereof.

### CROSS-REFERENCE TO RELATED APPLICATIONS

This application claims the benefit of U.S. Provisional Application 63/239,714 filed 1 Sep. 2021; which is incorporated herein by reference in its entirety for all purposes.

### STATEMENT REGARDING FEDERALLY SPONSORED RESEARCH OR DEVELOPMENT

Not applicable.

### FIELD OF THE INVENTION

The present invention generally relates to methods and compositions for treatment of conditions associated with hyperproliferative cellular division, such as cancer, diabetes, pre-diabetes, conditions associated with diabetes or pre-diabetes, immune deficiency diseases and disorders, conditions associated with immune deficiency diseases and disorders, conditions associated with Alzheimer's disease and conditions associated with autoimmune diseases, such as lupus. BACKGROUND

Cancer is a term used for diseases in which abnormal cells divide without control in a given normal tissue. The disease process also involves invasion of adjacent tissues by these abnormal cells, and spread of these abnormal cells to regional lymph nodes and to distant sites (metastasis) via the circulatory (blood) and immune (lymph) systems. Cancer can affect just about every organ in the human body. There are more than 100 different types of cancer, typically named for the organ or type of cell in which it starts.

Despite improved treatments for certain forms of cancer, it is still a leading cause of death in the United States. Reports of 2010 estimate that there will be more than 1.5 million new cases of cancer diagnosed (including non-melanoma skin cancers) and over 565,650 deaths from cancer in the U.S. in 2010.

Furthermore, some conventional treatments can exacerbate cancer conditions. Contrary to current thinking, the excision of lymph nodes in the axilla may tend to spread the cancer. The lymph nodes, rather than spreading malignancy through infected nodes, may be a sentinel for the immune system being due at least in part to various organs, and rather than spreading the cancer, actually plays a role in capturing and destroying malignant cells. Excising lymph nodes in the axilla, for example, may open up the possibility of lymphedema, whereas the removed lymph node(s) does nothing to fight cancer cells already spread elsewhere. As such, a more systemic treatment is needed.

A wide range of anti-neoplastic drugs have been identified for cancer therapy. Considerable difficulties, however, are encountered with the use of these drugs because of their toxicity to normal tissue. Monoclonal antibody technology brought with it a promising tool to attempt to specifically target cancer cells. Most of the clinical studies to date using this technology for cancer treatments have been disappointing, however, for many reasons, including the requirement for large doses to deliver sufficient drug to the cancer cells. Such large doses result in toxic side effects largely due to non-specific binding to normal cells and processing of the drugs by the liver. Thus, there exists a continuing need for safer and more effective treatments for control or elimination of conditions associated with hyperproliferative cellular division, such as cancer.

According to the guidelines of the American Diabetes Association, to be diagnosed with Type 2 diabetes, an individual must have a fasting plasma glucose level greater than or equal to 126 mg/dl or a 2-hour oral glucose tolerance test (OGTT) plasma glucose value of greater than or equal to 200 mg/dl. A related condition called pre-diabetes is defined as having a fasting glucose level of greater than 100 mg/dl but less than 126 mg/dl or a 2-hour OGTT plasma glucose level of greater than 140 mg/dl but less than 200 mg/dl. Mounting evidence suggests that the pre-diabetes condition may be a risk factor for developing cardiovascular disease. Prediabetes, also referred to as impaired glucose tolerance or impaired fasting glucose is a major risk factor for the development of type 2 diabetes mellitus, cardiovascular disease and mortality. Much focus has been given to developing therapeutic interventions that prevent the development of type 2 diabetes by effectively treating prediabetes.

The global health crisis of diabetes and pre-diabetes has been well established. The prevalence of type 2 diabetes and pre-diabetes is reaching pandemic proportions world-wide, and their prevalence is expected to continue to rise in the next two decades, further exacerbating the current world wide health crisis surrounding these diseases as estimates of people diagnosed with diabetes will likely exceed 350 million globally by 2030. Diabetes and its associated co-morbidity continue to exact an exceptionally high toll on both patients and the healthcare system. In the United States, diabetes represents 11% of the US health care expenditure with cardiovascular disease accounting for approximately 20% of the annual direct medical costs for diabetes. Despite the concerted effort to reduce cardiovascular risk factors in patients with diabetes, sixty-five percent of patients with diabetes will die from heart disease and stroke and the fact remains that type 2 diabetes increases the risk for cardiovascular disease two fold for men and three fold for women relative to gender matched individuals without type 2 diabetes. A safe and effective treatment for these disorders would impart unparalleled significant benefit to humanity.

AIDS, or Acquired Immunodeficiency Syndrome, one of the best studied immune deficiency diseases and disorders, is caused by the human immunodeficiency virus (HIV) and is characterized by several clinical features, including wasting syndromes, central nervous system degeneration and profound immunosuppression that results in opportunistic infections and malignancies. HIV is a member of the lentivirus family of animal retroviruses, which include the visna virus of sheep and the bovine, feline and simian immunodeficiency viruses (SIV). Two closely related types of HIV, designated HIV-1 and HIV-2, have been identified thus far, of which HIV-1 is by far the most common cause of AIDS. However. HIV-2, which differs in genomic structure and antigenicity, causes a similar clinical syndrome.

The AIDS epidemic is taking a devastating toll in human lives. According to the World Health Organization, almost 40 million people were living with HIV at the end of 2006, a year in which 4.3 million people were newly infected and 2.9 million died of AIDS-related diseases. Most new infections are occurring in the developing world, where women are most vulnerable. In sub-Saharan Africa, for example, 57% of people living with HIV are women, and young women between 15 and 24 years old are at least three times more likely to be HIV positive than young men.

Destruction of CD4+ T-lymphocytes, which are critical to immune defense, is a major cause of the progressive immune dysfunction that is the hallmark of AIDS disease progression. The loss of CD4+ T cells seriously impairs the body's ability to fight most invaders, but it has a particularly severe impact on the defenses against viruses, fungi, parasites and certain bacteria, including mycobacteria.

The ability to elicit broad and potent neutralizing antibodies against HIV-1 is a major challenge in the development of an HIV-1 vaccine. Namely, HIV-1 has evolved an impressive array of strategies to evade antibody-mediated neutralization. Broadly Neutralizing Antibodies (bNAbs) develop over time in a proportion of HIV-1 infected individuals, and a handful of broad neutralizing monoclonal antibodies have been isolated from clade B infected donors. These antibodies tend to display less breadth and potency against non-clade B viruses, and they recognize epitopes on the virus that so far have failed to elicit broad neutralizing responses when incorporated into a diverse range of immunogens.

Therefore, methods and compositions that stimulate the immune system of HIV patients would represent a significant advance in the art.

Alzheimer's disease (AD) is a progressive mental deterioration and form of dementia that often occurs in old age due to generalized degeneration of the brain. AD is a neurodegenerative disorder that affects memory and other cognitive functions, and is the most common cause of dementia. An Alzheimer's Disease Association (ADA) survey shows that 5.4 million people in the United States (US) currently have AD and 13.5 million are expected to have AD within the next 40 years. AD affects over 26 million people worldwide and currently there is no cure for the disease. With the growing number of people living to older ages, there is an urgency to find methods and compositions useful in the treatment and prevention of AD.

Detecting and diagnosing immune-mediated disorders, such as autoimmune disorders, is challenging, with patients having a difficult time receiving an accurate or correct diagnosis. Autoimmune diseases remains a major cause of morbidity and mortality. In many instances, patients are often misdiagnosed with other autoimmune conditions because of the closely related nature of these diseases. Autoimmune disease patients can experience chronically active disease, fluctuating rounds of remission and flare, or long quiescence. Accurately detecting and determining the status of a patient is central to prescribing appropriate drug regimens, evaluating treatment outcomes, defining patient subgroups, and early detection of flare onsets in order to improve therapeutic outcomes of patients afflicted with an autoimmune disease. Examples of autoimmune diseases include, but are not limited to, systemic lupus erythematosus (SLE), rheumatoid arthritis, Sjogren's disease, multiple sclerosis, ulcerative colitis, psoriatic arthritis, scleroderma and/or type I diabetes. Compositions and methods for the treatment of autoimmune diseases would represent a significant advance in the art.

### SUMMARY OF THE INVENTION

The present invention provides therapeutic agents, and methods for their manufacture and use for treatment of conditions associated with hyperproliferative cellular division, such as cancer, diabetes, pre-diabetes, or with conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases.

One aspect is directed to a composition for the treatment of conditions associated with hyperproliferative cellular division, diabetes or pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases. In various embodiments, such composition comprises an extract of a biological sample (e.g., serum or plasma) of an animal inoculated with an antigenic agent, wherein the extract exhibits cytotoxic activity towards cells exhibiting conditions associated with hyperproliferative cellular division, or exhibits enhancement of beta cells in diabetes or pre-diabetes, or conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases. In various embodiments, the antigenic agent is a virus, bacteria, protozoa, or fungi. In some embodiments, the antigenic agent comprises a fungi. In some embodiments, the antigenic agent is an Ascomyceta. In one embodiment, the antigenic agent is an Aspergillus. In various configurations, the Aspergillus is one or more of Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus israelii, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowi, Aspergillus tamari, Aspergillus terreus, Aspergillus ustus, or Aspergillus versicolor. In another embodiment, the antigenic agent comprises at least two of a virus, bacteria, protozoa, or fungi. In one configuration, the antigenic agent further comprises an Actinomyces, such as Actinomyces bovis. In some embodiments, the actinic agent is a macerated actinic agent. In some embodiments, the composition further comprises a pharmaceutically acceptable carrier or excipient.

Another aspect is directed to a method of producing a composition for treatment of conditions associated with hyperproliferative cellular division, diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases. In various embodiments, such method comprises inoculating an animal with an antigenic agent comprising a virus, bacteria, protozoa, or fungi; obtaining a biological fluid from the inoculated animal; extracting the biological fluid with an organic solvent to produce an aqueous phase and an organic phase; and isolating the organic phase; wherein the organic phase comprises an anti-cancer composition that exhibits cytotoxic activity towards cells exhibiting conditions associated with hyperproliferative cellular division, or an anti-diabetes or anti-pre-diabetes composition that exhibits enhancement of beta cells in diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases. In one embodiment, the antigenic agent comprises a fungi. In some embodiments, the antigenic agent is an Ascomyceta. In one embodiment, the antigenic agent is an Aspergillus. In various configurations, the Aspergillus is one or more of Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus israelii, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowi, Aspergillus tamari, Aspergillus terreus, Aspergillus ustus, or Aspergillus versicolor. In another embodiment, the antigenic agent comprises at least two of a virus, bacteria, protozoa, or fungi. In one configuration, the antigenic agent further comprises an Actinomyces, such as Actinomyces bovis. In some embodiments, the actinic agent is a macerated actinic agent. In some embodiments, the method further comprises macerating the actinic agent.

In some embodiments, the organic solvent comprises one or more solvents selected from the group consisting of hydrocarbons, halogenated hydrocarbons, alcohol, ketone, ester, and ether. In one embodiment, the solvent hydrocarbon is selected from the group consisting of n-hexane, n-heptane, petroleum ether, and cyclohexane. In one embodiment, the solvent halogenated hydrocarbon is selected from the group consisting of chloroform, dichloromethane and carbon tetrachloride. In one embodiment, the solvent alcohol is selected from the group consisting of methanol, ethanol, propanol and butanol. In one embodiment, the solvent ester is ethyl acetate. In one embodiment, the solvent ether is diethyl ether.

In various embodiments, the method comprises at least two extraction steps. In some embodiments, the method comprises at least three extraction steps. In one embodiment, the method comprises a first extraction with ether, a second extraction with benzene, and a third extraction with water. Extraction can be preceded or followed with other separation techniques, such as ammonium sulfate fractionation.

Another aspect is directed to a method of treating a condition associated with hyperproliferative cellular division, or diabetes, pre-diabetes, a condition associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases in a subject. In various embodiments, the method comprises administering a therapeutically effective amount of a composition described above to a subject in need thereof. In various embodiments, the method comprises administering a therapeutically effective amount of a composition produced according to methods described above to a subject in need thereof.

In various embodiments, the condition associated with hyperproliferative cellular division is selected from the group consisting of a solid cancer and a hematopoietic malignant cancer. In some embodiments, the solid cancer is selected from the group consisting of gastrointestinal tumors, cancer of liver and biliary tract, pancreatic cancer, prostatic cancer, testicular cancer, lung cancer, breast cancer, malignant melanoma, ovarian cancer, uterine cancer, cervical cancer, cancer of the head and neck, bladder cancer, sarcomas and osteosarcomas, Kaposi sarcoma, AIDS-related Kaposi sarcoma and renal carcinoma. In some embodiments, the subject is diagnosed with cancer or diagnosed as at risk for cancer.

In other embodiments, the condition associated with diabetes or pre-diabetes is cardiovascular disease. In further embodiments, the subject is diagnosed with diabetes or pre-diabetes, or diagnosed as at risk for diabetes or pre-diabetes, or diagnosed with an immune deficiency disease or disorder, including HIV, Alzheimer's disease or conditions associated with Alzheimer's disease, or an autoimmune disease, such as systemic lupus erythematosus (SLE), rheumatoid arthritis, Sjogren's disease, multiple sclerosis, ulcerative colitis, psoriatic arthritis, scleroderma and/or type I diabetes, or conditions associated with such autoimmune diseases.

In various embodiments, administration is parenteral, pulmonary, oral, topical, intradermal, intramuscular, intraperitoneal, intravenous, subcutaneous, intranasal, epidural, ophthalmic, buccal, or rectal administration.

In various embodiments, the composition is substantially non-toxic in non-cancerous cells or beta cells. In some embodiments, the administration of the composition results in minimal to no side effects in the subject.

In various embodiments, the biological sample includes one or more of whole blood, plasma, serum, pleural fluid, cerebrospinal fluid, or culture fluid. In some embodiments, the biological sample includes one or more of plasma or serum.

Another aspect is directed to a kit for treatment of a condition associated with hyperproliferative cellular division, diabetes, pre-diabetes, a condition associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases. In various embodiments, the kit comprises a composition described above. In various embodiments, the kit comprises a composition produced according to methods described above.

A further aspect is directed to the compositions described herein being used in microfluidic platforms or “chips” for testing and understanding cancer, diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a Total Ion Chromatogram (TIC) from BSTFA-derivatized glass ampule (GA) (red) and plastic bottle (PB) (green) samples as well as the underivatized PB sample.

FIG. 2 is an expanded view on the later eluting peaks. The y-axes are not the same scale.

FIG. 3 is a mass spectrum of β-Sitosterol trimethylsilyl ether

FIG. 4 is an ESI-MS spectrum from GA sample.

FIG. 5 is an expanded view of a region of the spectrum in FIG. 4 showing region around m/z 923.93. The predicted spectra for the predicted empirical formula C₂₅H₆₇N₁₈O₁₉ is shown in FIG. 6.

FIG. 6 is the predicted spectrum based on empirical formula C₂₅H₆₇N₁₈O₁₉.

FIG. 7 is a Table of proteins identified in the GA sample.

FIG. 8 is a Table of proteins identified in the PB sample.

FIG. 9 is an image showing the two samples, the Glass Ampoule (GA) on left and Plastic Bottle (PB) on right.

FIG. 10 is an image of a Sypro gel test used to analyze the two samples (GA=glass ampoule, PB=plastic bottle). The faint horizontal bands correspond to proteins.

### DETAILED DESCRIPTION OF THE INVENTION

Provided herein are compositions, kits, and methods for the treatment of conditions associated with hyperproliferative cellular division, such as cancer, diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases. The present invention is based, at least in part, on the discovery of a therapeutic substance having anti-cancer properties and beta cell enhancement properties isolated from a large animal, such as a horse or a cow, inoculated with a filtrate resulting from maceration of an Aspergillus culture solution.

It has been discovered that the stimulation of the reticuloendothelial system of an animal, such as a horse, can be accomplished by injecting into the animal a culture containing an appropriate antigenic agent. In various embodiments, the antigenic agent comprises a bacteria or fungus, preferably, a fungus, more preferably an Ascomyceta, and most preferably Aspergillus. An antigenic agent can be delivered via a filtrate resulting from, for example, maceration of a culture solution comprising the agent of interest. The purpose of inoculation is not to infect the animal with the agent; but rather, to stimulate the reticuloendothelial system of the animal to produce and secrete in the blood stream of the animal a biologically active substance having anti-hyperproliferative cellular division effects, anti-diabetes or pre-diabetes effects, anti-immune deficiency diseases and disorders or conditions associated with immune deficiency diseases and disorders effects, anti-Alzheimer's disease or conditions associated with Alzheimer's disease effects, or anti-autoimmune disease or conditions associated with autoimmune diseases effects, as described herein. After inoculation of an animal with an antigenic agent, a composition comprising a biologically active substance can be isolated from a biological fluid of the animal, for example from the serum or plasma of the animal.

It is presently thought by the inventors that cancer (or many forms thereof) is but one disease that expresses differently in different organs and tissues. It is also thought that cancer cells originate primarily from stem cells. Virus or bacteria are among the several factors thought to effect propagation of cancer cells from stem cells. Examples of viruses associated with human cancers include, but are not limited to, human papillomavirus, hepatitis B and hepatitis C virus, Epstein-Barr virus, and human T-lymphotropic virus. Examples of bacteria associated with human cancers include, but are not limited to, Helicobacter pylori. Various embodiments of the composition described herein are thought to regulate or interfere with propagation of cancer cells from parent cancer cells as well as, ultimately, stem cells. Additionally, when IL33 enters cancer cells it interferes with the ability of the immune system of the body to recognize the malignant cells as cancerous and destroy them, leading to increased metastasis. In certain embodiments, the compositions are thought to cross the cell membrane of malignant cells, destroying the cytoplasm and mitochondria, and thereby destroying the malignant cells and eliminating IL33, thus preventing the malignant cells from proliferating, which would lead to metastasis.

In addition, the link between diabetes or pre-diabetes, immune deficiency diseases, Alzheimer's disease and autoimmune diseases and cancer has been established in recent years. The presently disclosed compositions are thought to heighten the immune system by particularly enhancing beta cells, causing them to strengthen specific anti-cancer antibodies. Thus, various embodiments of the composition described herein are thought to treat diabetes or pre-diabetes, conditions associated with diabetes or pre-diabetes, immune deficiency diseases and disorders, conditions associated with immune deficiency diseases and disorders, Alzheimer's disease and conditions associated with Alzheimer's disease, and autoimmune disease and conditions associated with autoimmune diseases via this enhancement of beta cells.

The compositions described herein can be used in microfluidic platforms or “chips” for testing and understanding cancer, diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases. Briefly, tissues associated with cancer, diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases are placed in a multicellular, layered, microfluidic culture, and the effect of the compositions described herein on interactions between the tissues and the compositions are studied.

Use of these “chips” permits the recreation of various human tumor microenvironments in vitro, allowing for mechanistic studies of cancer cell behavior and drug efficacy and safety, such as the efficacy of the compositions described herein. In other examples pancreatic tissue that generates insulin is studies to determine the ability of the diabetes compromised pancreatic cells to produce insulin in the presence of the presently described compositions. Additionally, compromised immune cells can be placed in such “chips”, and the effect of the presently described compositions on the ability of the compromised immune cells to produce antibodies is studied. Furthermore, brain tissue that generates β-amyloid protein or peptides can be placed in such “chips” to study the ability of the brain cells to decrease the production of β-amyloid protein or peptides in the presence of the presently described compositions. Finally, tissue that generates autoantibodies can be placed in such “chips” to study the effect of the compositions described herein on the ability of the tissue to decrease or cease the production of autoantibodies.

### Antigenic Agent

As described above, various aspects of the invention include compositions formed via introduction of an antigenic agent into an organism having an immune system and subsequent isolation of a biological fluid of the animal and extraction of a composition comprising a biologically active agent having anti-hyperproliferative cellular division properties or anti-diabetes or anti-pre-diabetes properties, anti-immune deficiency disease or disorder, including HIV, properties, anti-Alzheimer's disease or conditions associated with Alzheimer's disease properties, or anti-autoimmune disease or conditions associated with autoimmune diseases properties.

In various embodiments, the antigenic agent comprises a virus, bacteria, protozoa, or fungi. Preferably, the antigenic agent is an Ascomyceta, more preferably Aspergillus spp. For example, the antigenic agent can be one or more of Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus israelii, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowi, Aspergillus tamari, Aspergillus terreus, Aspergillus ustus, and Aspergillus versicolor.

In some embodiments, the antigenic agent can comprise further virus, bacteria, protozoa, or fungi. For example, the actinic agent can include both Aspergillus spp. and Actinomyces spp (e.g., Actinomyces bovis).

The antigenic agent can be prepared by, for example, maceration in a saline solution with an optionally cytolytic agent being included. The actinic agent can be administered as a live agent or a killed agent. The actinic agent is preferably a killed actinic agent. The actinic agent can be administered as a sterile composition. It is understood that, in some embodiments, because the purpose of inoculation is not infection but rather stimulation of the reticuloendothelial system of the animal, it is preferred that the antigenic agent be prepared in a non-infectious form for inoculation.

### Source Animal

A composition described herein can be isolated from an animal inoculated with an antigenic agent. The inoculated animal can optionally receive a fresh blood transfusion to increase or prolong the defensive immune response. The animal can be selected from mammals, reptiles, and avians, more preferably a horse, gorilla, monkey, cow, sheep, pig, dog, cat, rabbit, rat, mouse, or chicken. The animal can be a male animal or a female animal, where female animals are preferred. It is presently thought that progesterone plays a role. Preferably, a composition is isolated from an animal not susceptible to infection by the actigenic agent. For example, a cow can be susceptible to lumpy jaw, a granulomatous non-cancerous tumor, where Actinomyces bovis is present. Most preferably, a composition is isolated from a horse, such as an Arabian horse. The Arabian horse can be full-blooded or part-blooded, and is preferably a full-blooded Arabian horse, and more preferably a full-blooded female Arabian horse.

### Isolation and Extraction

After inoculation, a biological fluid of the inoculated animal can be obtained. Collection of biological fluid from the inoculated animal can be according to methods commonly understood in the art. Likewise, extraction of the biological fluid can be according to methods commonly understood in the art.

The biological fluid of the inoculated animal can be, for example, whole blood, plasma, serum, pleural fluid, cerebrospinal fluid, culture fluid, or other localized body fluid. Preferably the biological fluid is plasma or serum, or some component thereof, such as globulin or albumin. For example, the biological sample from which an active agent is isolated can comprise a globulin. The balance of discussion is directed to plasma and serum but one of ordinary skill in the art will understand that such procedures can be adapted for other biological fluids.

A fluid sample, such as serum or plasma, from the inoculated animal can be obtained after a period of time sufficient to develop an agent with active biological activity as described herein. For example, serum can be obtained from an animal about two weeks, about three weeks, about four weeks, about five weeks, about six weeks, about seven weeks, or about eight weeks, or more, after inoculation. As another example, plasma can be obtained from an animal about two weeks, about three weeks, about four weeks, about five weeks, about six weeks, about seven weeks, or about eight weeks, or more, after inoculation. Preferably, serum or plasma is obtained from an animal from about three weeks to about six weeks after inoculation.

The extraction process of the biological fluid from the inoculated animal can be by mixing, swirling, vortexing, rotating or by any other suitable process. The extraction time should be sufficient to allow the solvent to solubilize all, or substantially all, desired active ingredients of the sample and will vary with the extraction process. For example, vortexing can occur over a period of about 30 to about 60 minutes and typically about 30 minutes. In some embodiments, extraction protocols can isolate greater than about 60% of the active agent. For example, extraction protocols can isolate at least about 65%, at least about 70%, at least about 75%, at least about 80%, at least about 85%, at least about 90%, at least about 95%, or at least about 99% of the active agent. In some embodiments, a plasma or serum sample of an inoculated animal is a clear yellowish fluid and an isolated extract containing active agent comprises a straw or yellowish color.

The method may include the further step of concentrating one or more phases to produce a concentrate or otherwise isolating a desired active agent, for example, by lyophilization or by any other technique which would be familiar to the person skilled in the art.

An extract can be prepared using an organic solvent. Exemplary solvents include, but are not limited to, hydrocarbons, halogenated hydrocarbons, alcohol, ketone, ester, and ether. Examples of a solvent hydrocarbon include, but are not limited to, n-hexane, n-heptane, petroleum ether, and cyclohexane. Examples of a solvent halogenated hydrocarbon include, but are not limited to, chloroform, dichloromethane and carbon tetrachloride. Examples of a solvent alcohol include, but are not limited to, methanol, ethanol, propanol and butanol. An example of a solvent ester includes, but is not limited to, ethyl acetate. An example of a solvent ether includes, but is not limited to, diethyl ether.

The extraction protocol can comprise a series of extraction steps with one or more solvents. For example, an ether extract of citrated or heparinized plasma can be made, producing ether soluble materials. As another example, a benzene extract of plasma, or some prior extraction production, can be made, producing benzene soluble materials. As another example, a water extract of plasma, or some prior extraction production, can be made, producing water soluble materials. In one embodiment a series of extractions from plasma including ether, benzene, and water are made, producing ether-benzene-water soluble materials from plasma. Such extraction product, or other extraction products, can be further extracted with, for example, redistilled anhydrous ether, redistilled benzene, or anhydrous redistilled methanol. Such sequential extractions can remove materials trapped in fat during the ether and benzene extractions and which are soluble in water. Various combinations of extractions can be made.

An extraction product can be placed in a physiologically suitable oil, such as mineral oil or sesame oil. Alternatively, an extraction product can be dissolved in physiological saline. Alternatively, an extraction product can be evaporated to produce residue.

An extract can be isolated by a variety of means. For example, a composition can be isolated via ammonium sulfate fractionation (e.g., Kohl method of ammonium fractionation). As another example, an extract can be isolated from a sample by organic solvent(s). The extract can be isolated by a series of organic solvents. An organic solvent extract can be evaporated to produce a powder or crystalline form of the composition. A water extract can be evaporated to produce a powder or crystalline form of the composition. Compositions comprising an active agent are further described below. In some embodiments, isolation methods produce a composition having at least about 60% purity. For example, isolation methods can produce a composition having at least about 65%, at least about 70%, at least about 75%, at least about 80%, at least about 85%, at least about 90%, at least about 95%, or at least about 99% purity. For example, a Kohl method of ammonium fractionation can isolate a composition having at least about 95% purity.

### Composition

One aspect of the invention provides a composition for the treatment of conditions associated with hyperproliferative cellular division, diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases. Such composition comprises an active biological agent having antihyperproliferative cellular division properties and/or anti-diabetes or anti-pre-diabetes properties, anti-immune deficiency disease or disorder, including HIV, properties, anti-Alzheimer's disease or conditions associated with Alzheimer's disease properties, and/or anti-autoimmune disease or conditions associated with autoimmune diseases properties. In various embodiments, a composition is isolated and purified from a biological sample (e.g., plasma or serum) of an animal (e.g., a horse) inoculated with an antigenic agent (e.g., Aspergillus). In one embodiment, the composition described herein comprises an active biological agent isolated from a mammal that has been injected with a pre-treated serum.

It is currently thought that the therapeutic composition comprises one or more active biological agents selected from an immunopeptide (e.g., an antibody), a small molecule, a growth regulator (e.g., a growth inhibiting factor, GIF), or hormone or hormone-like molecule. In some embodiments, the therapeutic composition comprises a lipopolysaccharide, such as galacturonic acid, galactose, glucosamine, glucose, arabinose, or xylose, optionally in combination with a fat molecule. Compositions described herein may alter membrane permeability, inhibit bacterial cell wall synthesis, inhibit DNA translation and transcription (e.g., of the subject, viral, or bacterial), or inhibit essential metabolic synthesis, or a combination thereof.

A composition can be isolated from a biological sample of an inoculated animal (e.g., plasma or serum) by a variety of means. Any of the isolation or purification protocols described above in the context of an extract can be used to further isolate or purify one or more compositions from an extract. For example, a composition can be isolated via ammonium sulfate fractionation (e.g., Kohl method of ammonium fractionation). As another example, a composition can be extracted from a biological sample, such as serum or plasma, by organic solvent(s). The composition can be extracted by a series of organic solvents. An organic solvent extract can be evaporated to produce a powder or crystalline form of the composition. A solvated composition can be further purified by evaporation of the solvent and further extraction with water. A water extract can be evaporated to produce a powder or crystalline form of the composition. A dried form of the composition can be re-solubilized in, for example, water, saline, or a pharmaceutically acceptable oil (e.g., mineral oil). In some embodiments, a composition described herein is isolated via an automatic titration machine. In some embodiments, isolation methods produce a composition having at least about 60% purity. For example, isolation methods can produce a composition having at least about 65%, at least about 70%, at least about 75%, at least about 80%, at least about 85%, at least about 90%, at least about 95%, or at least about 99% purity.

Compositions described herein can be used in various therapeutic modalities for the treatment or prevention of conditions associated with hyperproliferative cellular division, such as cancer, diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases. Compositions described herein can be used in preventative modalities as a vaccine for the prevention of conditions associated with hyperproliferative cellular division, diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, or autoimmune disease and conditions associated with autoimmune diseases. It is understood that compositions described herein can function as ameliorative or curative factors for systemic or individual tumors, or ameliorative or curative factors for diabetes or pre-diabetes, for immune deficiency diseases and disorders, including HIV, or conditions associated with immune deficiency diseases and disorders, for Alzheimer's disease or conditions associated with Alzheimer's disease, or for autoimmune disease or conditions associated with autoimmune diseases.

Compositions described herein can act as a vaccine, in addition to acting as, for example, an ameliorative, a stabilizer for growth, or a curative factor. In some embodiments, a composition acts as an active vaccination, which can stimulate the body's immune system, inducing specific immunity to a particular antigen. It is thought that compositions described herein can stimulate the immune system by particularly enhancing beta cells. It is believed that beta cells strengthen anti-cancer antibodies. In some embodiments, administration of a composition acting as an active vaccination can result in immunity over the life of the subject. In some embodiments, a composition acts as an passive vaccination, in which injections containing antibodies formed in another person or animal may last one or more months because the antibodies disappear and do not stimulate the immune system.

In some embodiments, the therapeutic composition can comprise a small molecule. A preferable small molecule having activity as described herein may encompass numerous chemical classes, though typically they are organic molecules, preferably small organic compounds having a molecular weight of more than 50 and less than about 2,500 Daltons. A small molecule of the therapeutic composition may comprise functional groups for structural interaction with proteins, particularly hydrogen bonding, and can include at least an amine, carbonyl, hydroxyl or carboxyl group, preferably at least two of the functional chemical groups. A small molecule of the therapeutic composition may comprise cyclical carbon or heterocyclic structures and/or aromatic or polyaromatic structures substituted with one or more of the above functional groups.

A small molecule of the therapeutic composition described herein may include a lead-like compound or a drug-like compound. A lead-like compound is generally understood to have a relatively smaller scaffold-like structure (e.g., molecular weight of about 150 to about 350 Daltons) with relatively fewer features (e.g., less than about 3 hydrogen donors and/or less than about 6 hydrogen acceptors; hydrophobicity character xlogP of about −2 to about 4) (see, e.g., Angewante (1999) Chemie Int. ed. Engl. 24, 3943-3948). In contrast, a drug-like compound is generally understood to have a relatively larger scaffold (e.g., molecular weight of about 150 to about 500 Daltons) with relatively more numerous features (e.g., less than about 10 hydrogen acceptors and/or less than about 8 rotatable bonds; hydrophobicity character xlogP of less than about 5) (see, e.g., Lipinski (2000) J. Pharm. Tox. Methods 44, 235-249).

A small molecule of the therapeutic composition described herein may have one or more “drug-like” characteristics, such as those summarized into the four rules of Lipinski (generally known as the “rules of fives” because of the prevalence of the number 5 among them). The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å. It will be understood that a small molecule of the therapeutic composition may not meet all, or even any, of these characterizations. Nonetheless, the above guidelines are helpful in screening and design of various therapeutic compositions described herein.

In some embodiments, the therapeutic composition can comprise a hormone or hormone-like substance. It is presently thought that such hormone or hormone-like substance may be produced in a gland of an animal inoculated with an antigenic agent described herein. For example, a therapeutic composition described herein may comprise a hormone or hormone-like substance produced in the anterior pituitary. As another example, a therapeutic composition described herein may comprise a hormone or hormone-like substance produced in the pineal gland. Such a hormone or hormone-like substance may be related to, derived from, or supplement the function of other pineal gland-related hormones, such as melatonin or serotonin. A hormone or hormone-like substance contained in a therapeutic composition described herein and produced in the pineal gland may, at least in part, contribute to regulation of the circadian rhythm, which is thought to be a significant predictor of survival in cancers, such as breast cancer (see, e.g., Mills et al. 2005 Journal of Pineal Research 39(4), 360; Navara and Nelson 2007 J. Pineal Res. 43(43), 215-224; Maestroni 1999 Adv Exp Med Biol 467, 217-226; Schernhammer et al. 2004 Cancer Epidemiol Biomarkers Prev 13(62), 936-943).

Various embodiments of the composition comprise a peptide or immunogenic substance isolated from an organism inoculated with an antigenic agent, as described above. In some embodiments, the composition comprises an antibody.

Antibodies within the scope of the invention include, for example, polyclonal antibodies, monoclonal antibodies (MAbs), antibody fragments (e.g., Fab, F(ab′)₂, F(ab′)₂, F(ab′)₃, Fc, single chain Fv (scFv), scFV-Fc, (scFv)₂, dsFv, Vh, Vl, Minibody, Diabody, Triabody, Tetrabody), and antibody fusion molecules. Engineering, production, purification, fragmentation, and use, including therapeutic use, of various types of antibodies are well known in the art (see generally, Carter (2006) Nat Rev Immunol. 6(5), 343-357; Teillaud (2005) Expert Opin Biol Ther. 5(Supp. 1) S15-27; Subramanian, ed. (2004) Antibodies : Volume 1: Production and Purification, Springer, ISBN 0306482452; Lo, ed. (2003) Antibody Engineering Methods and Protocols, Humana Press, ISBN 1588290921; Ausubel et al., ed. (2002) Short Protocols in Molecular Biology 5th Ed., Current Protocols, ISBN 0471250929; Brent et al., ed. (2003) Current Protocols in Molecular Biology, John Wiley & Sons Inc, ISBN 047150338X; Lo, ed. (2003) Antibody Engineering Methods and Protocols, Humana Press, ISBN 1588290921; Coligan (2005) Short Protocols in Immunology, John Wiley & Sons, ISBN 0471715786).

Polyclonal antibodies are heterogeneous populations of antibody molecules that are obtained from immunized animals, usually from sera. Polyclonal antibodies may be readily generated by one of ordinary skill in the art from a variety of warm-blooded animals, as well known in the art and described in the numerous references listed above. It is generally understood that larger mammals, such as a horse described herein, are preferred as the amount of serum that can be collected is greater. Generally, an antigen (as discussed above) is injected into the mammal (e.g., a horse). This can induce the p-lymphocytes to produce IgG immunoglobulins specific for the antigen. This polyclonal IgG can be purified from the mammal's serum.

Monoclonal antibodies are homogeneous populations of antibodies to a particular antigen. In contrast to polyclonal antibodies that may be specific for several epitopes of an antigen, monoclonal antibodies are usually specific for a single epitope. Generally, monoclonal antibodies are produced by removing β-cells from the spleen of an antigen-challenged animal (wherein the antigen includes the proteins described herein) and then fusing with myeloma tumor cells that can grow indefinitely in culture. The fused hybrid cells, or hybridomas, multiply rapidly and indefinitely and can produce large amounts of antibodies. The hybridomas can be sufficiently diluted and grown so as to obtain a number of different colonies, each producing only one type of antibody. The antibodies from the different colonies can then be tested for their ability to bind to the antigen, followed by selection of the most effective. In particular, monoclonal antibodies can be obtained by any technique that provides for the production of antibody molecules by continuous cell lines in culture such as those described in references listed above.

Such antibodies can be of any immunoglobulin class including IgG, IgM, IgE, IgA, IgD and any subclass thereof. Preferably, the antibody is of the IgE immunoglobulin class. In some embodiments, the antibody is of the IgG immunoglobulin class, which can be utilized either in isolation or in combination with IgE. A hybridoma producing a mAb of the invention may be cultivated in vitro or in vivo. The ability to produce high titers of mAbs in vivo makes this a particularly useful method of production. MAbs generally have a longer terminal half-life than many antibody fragments, translating into greater uptake, that can be desirable for therapeutic applications.

MAbs can be selected on the basis of their (a) specificity, (b) high binding affinity, (c) isotype, and (d) stability. MAbs can be screened or tested for specificity using any of a variety of standard techniques, including Western Blotting (Koren, et al., Biochim. Biophys. Acta 876:91-100 (1986)) and enzyme-linked immunosorbent assay (ELISA) (Koren, et al., Biochim. Biophys. Acta 876:91-100 (1986)).

Chimeric, humanized, and fully human MAbs can effectively overcome potential limitations on the use of antibodies derived from non-human sources to treat cancer, thus providing decreased immunogenicity with optimized effector functions (see e.g., Teillaud (2005) Expert Opin. Biol. Ther. 5(1), S15-S27; Tomizuka et al. (2000) Proc. Nat. Acad. Sci. USA 97, 722-727; Carter et al. (2006) Nat Rev Immunol. 6(5), 343-357, 346-347).

It may be desirable to produce and use functional antibody fragments (e.g., Fab, F(ab′)₂, F(ab′)₂, F(ab′)₃, Fc, single chain Fv (scFv), scFV-Fc, (scFv)₂, dsFv, Vh, Vl, Minibody, Diabody, Triabody, Tetrabody). Generally, these alternative antibody formats can span a molecular-weight range of 12-150 kDa; a valency (n) range from monomeric (n=1), dimeric (n=2), trimeric (n=3), tetrameric, or even higher; and antigen-binding specificities from one to more than three antigens or epitopes on the same antigen.

Preferably, an antibody is a monoclonal antibody or antibody fragment specific for any of the above targets, and will usually bind with at least a K_{D} of about 1 mM, more usually at least about 300 μM, typically at least about 30 μM, preferably at least about 10 μM, and more preferably at least about 3 μM or better.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent having anti-hyperproliferative cellular division properties, anti-diabetes or pre-diabetes properties, anti-immune deficiency diseases and disorders, including HIV, properties, anti-Alzheimer's disease or conditions associated with Alzheimer's disease properties, or anti-autoimmune disease or conditions associated with autoimmune diseases properties, preferably in purified form, together with a suitable amount of carrier so as to provide the form for proper administration to the subject.

The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other chemical or physical forces, such as magnetic or electronic/al forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents described herein can also be used in combination with other therapeutic modalities, as described further below. Thus, in addition to the therapies described herein, one may also provide to the subject other therapies known to be efficacious for treatment of conditions associated with hyperproliferative cellular division, diabetes, pre-diabetes, conditions associated with diabetes or pre-diabetes, immune deficiency diseases and disorders, including HIV, conditions associated with immune deficiency diseases and disorders, Alzheimer's disease or conditions associated with Alzheimer's disease, or autoimmune disease or conditions associated with autoimmune diseases.

### Method of Treatment

Thus is provided therapeutic methods directed against conditions associated with hyperproliferative cellular division, diabetes, pre-diabetes, conditions associated with diabetes or pre-diabetes, immune deficiency diseases and disorders, including HIV, conditions associated with immune deficiency diseases and disorders, Alzheimer's disease and conditions associated with Alzheimer's disease, and autoimmune disease and conditions associated with autoimmune diseases or disorders. Briefly, therefore, one aspect of the present invention is directed to a process of treating conditions associated with hyperproliferative cellular division, diabetes, pre-diabetes, conditions associated with diabetes or pre-diabetes, immune deficiency diseases or disorders, including HIV, conditions associated with immune deficiency diseases and disorders, Alzheimer's disease and conditions associated with Alzheimer's disease, and autoimmune disease and conditions associated with autoimmune diseases, in a subject, the process comprising administering a composition described herein to a subject in need thereof. A subject in need of the therapeutic methods described herein can be diagnosed with having a condition associated with hyperproliferative cellular division, diabetes, pre-diabetes, a condition associated with diabetes or pre-diabetes, immune deficiency diseases and disorders, including HIV, conditions associated with immune deficiency diseases and disorders, Alzheimer's disease or conditions associated with Alzheimer's disease, or autoimmune disease or conditions associated with autoimmune diseases, or at risk thereof. For example, a subject can be diagnosed with having or at risk for having cancer according to the Schandl Cancer Profile blood panel test, the Navarro Test, or the AMAS test, or diagnosed with having or at risk for having diabetes or pre-diabetes according to fasting plasma glucose levels or a 2-hour oral glucose tolerance test.

Conditions associated with hyperproliferative cellular division refer generally to any clinical condition characterized by, or otherwise involving, an increased rate of cell division relative to a normal reference rate. Conditions associated with hyperproliferative cellular division include benign and malignant conditions. Malignant conditions associated with hyperproliferative cellular division generally include one or more of uncontrolled growth (i.e., division beyond the normal limits), invasion (i.e., intrusion on and destruction of adjacent tissues), and metastasis (i.e., spread to other locations in the body via lymph or blood). Conditions associated with hyperproliferative cellular division include, but are not limited to, cancer, myeloproliferative syndromes such as Langerhans cell histiocytosis, mastocytosis, mixed myeloproliferative and myelodysplastic conditions; and dermal proliferative conditions such as psoriasis, non-bullous congenital ichthyosiform erythroderma. In preferred embodiments, the condition associated with hyperproliferative cellular division is cancer, including tumor-forming cancers and non-tumor forming cancers (e.g., hematopoietic malignant cancers).

Cancer types generally include carcinoma (malignant tumors derived from epithelial cells, including breast, prostate, lung and colon cancer); sarcoma (malignant tumors derived from connective tissue, or mesenchymal cells); lymphoma and leukemia (malignancies derived from hematopoietic cells); germ cell tumor (tumors derived from totipotent cells); cancer stem cells, blastic tumor or blastoma (tumor resembling an immature or embryonic tissue).

Cancers include, but are not limited to, gastrointestinal tumors, cancer of liver and biliary tract, pancreatic cancer, prostatic cancer, testicular cancer, colorectal cancer, lung cancer, breast cancer, cutaneous melanoma, ovarian cancer, uterine cancer, cervical cancer, endometrial cancer, cancer of the head and neck, brain, bladder cancer, leukemia, non-Hodgkin lymphoma, sarcomas and osteosarcomas, Kaposi sarcoma, AIDS-related Kaposi sarcoma, and renal carcinoma.

The compositions described herein may interfere with one or more characteristic biological properties of malignant tumor cells including acquisition of self-sufficiency in growth signals, leading to unchecked growth; loss of sensitivity to anti-growth signals, also leading to unchecked growth; loss of capacity for apoptosis, in order to allow growth despite genetic errors and external anti-growth signals; loss of capacity for senescence, leading to limitless replicative potential (immortality); acquisition of sustained angiogenesis, allowing the tumor to grow beyond the limitations of passive nutrient diffusion; acquisition of ability to invade neighboring tissues, the defining property of invasive carcinoma; acquisition of ability to build metastases at distant sites, the classical property of malignant tumors (carcinomas or others); and loss of capacity to repair genetic errors, leading to an increased mutation rate (genomic instability), thus accelerating all the other changes. In some embodiments, compositions described herein can be used as a stabilizing factor, a regressive factor, or overall cure. For example, a composition can be administered to a subject prior to diagnosis of malignancy. As another example, a composition can be administered to a subject upon diagnosis of a benign condition so as to hold back metastatic progression (e.g., uterine fibroids). As another example, a composition can be administered to a subject diagnosed with a malignant condition as a regressive factor to decrease tumor size or growth rate. As another example, a composition can be administered to a subject diagnosed with a malignant condition so as to eliminate the malignancy and provide a cure for the condition.

Conditions associated with diabetes or pre-diabetes refer generally to any clinical condition characterized by, or otherwise involving, an increased level of plasma glucose relative to a normal reference rate. Conditions associated with diabetes or pre-diabetes include cardiovascular disease.

A composition can be administered to a subject prior to diagnosis of diabetes or pre-diabetes. As another example, a composition can be administered to a subject upon diagnosis of pre-diabetes so as to hold back diabetes progression. As another example, a composition can be administered to a subject diagnosed with diabetes or pre-diabetes so as to eliminate the condition and provide a cure for the condition.

Conditions associated with immune deficiency diseases or disorders refer generally to any clinical condition characterized by, or otherwise involving, a decreased level of immune cells or antibodies relative to a normal reference rate. Examples of primary immune deficiency disorders include X-linked agammaglobulinemia (XLA), common variable immunodeficiency (CVID), and severe combined immunodeficiency (SCID). Conditions associated with immune deficiency diseases or disorders include secondary immunodeficiency disorders, including cancers of the immune system, such as leukemia, immune-complex diseases, such as viral hepatitis, and multiple myeloma, and increased susceptibility to bacterial, viral or fungal infections.

A composition can be administered to a subject prior to diagnosis of immune deficiency disease or disorder, including HIV. As another example, a composition can be administered to a subject upon diagnosis of early stage immune deficiency disease or disorder, including HIV, so as to hold back progression of such immune deficiency disease or disorder. As another example, a composition can be administered to a subject diagnosed with an immune deficiency disease or disorder, including HIV, or conditions associated with an immune deficiency disease or disorder so as to eliminate the condition and provide a cure for the condition.

Conditions associated with Alzheimer's disease refer generally to any clinical condition characterized by, or otherwise involving, an increased level of β-amyloid protein or peptides relative to a normal reference rate.

A composition can be administered to a subject prior to diagnosis of Alzheimer's disease or a condition associated with Alzheimer's disease. As another example, a composition can be administered to a subject upon diagnosis of early stage Alzheimer's disease or a condition associated with Alzheimer's disease, so as to hold back progression of Alzheimer's disease or condition associated with Alzheimer's disease. As another example, a composition can be administered to a subject diagnosed with Alzheimer's disease or a condition associated with Alzheimer's disease, so as to eliminate the condition and provide a cure for the condition.

Conditions associated with autoimmune diseases refer generally to any clinical condition characterized by, or otherwise involving, an increased level of autoantibodies relative to a normal reference rate. Examples of autoimmune diseases include systemic lupus erythematosus (SLE), rheumatoid arthritis, Sjogren's disease, multiple sclerosis, ulcerative colitis, psoriatic arthritis, scleroderma and/or type I diabetes.

A composition can be administered to a subject prior to diagnosis of an autoimmune disease or a condition associated with an autoimmune disease. As another example, a composition can be administered to a subject upon diagnosis of an early stage autoimmune disease or a condition associated with autoimmune disease, so as to hold back progression of the autoimmune disease or condition associated with autoimmune disease. As another example, a composition can be administered to a subject diagnosed with an autoimmune disease or a condition associated with an autoimmune disease, so as to eliminate the condition and provide a cure for the condition.

Methods described herein are generally performed on a subject in need thereof. A determination of the need for treatment will typically be assessed by a history and physical exam consistent with the condition associated with hyperproliferative cellular division at issue, diabetes, pre-diabetes, the condition associated with diabetes or pre-diabetes, immune deficiency disease or disorder, including HIV, conditions associated with immune deficiency disease or disorder, Alzheimer's disease or conditions associated with Alzheimer's disease, or autoimmune disease or conditions associated with autoimmune diseases. Diagnosis can be facilitated by, for example, blood tests, X-rays, CT scans, endoscopy, thermography, or biopsy. Preferably, a subject has a biopsy and histological or cytogenetic examination of a cell sample by a pathologist, or a blood test. Diagnosis of the various conditions treatable by the methods described herein is within the skill of the art. Subjects with an identified need of therapy include those with a diagnosed condition associated with hyperproliferative cellular division or at risk for a condition associated with hyperproliferative cellular division, diabetes, pre-diabetes, a condition associated with diabetes or pre-diabetes or at risk for developing diabetes or pre-diabetes, immune deficiency disease or disorder, including HIV, or conditions associated with immune deficiency disease or disorder, Alzheimer's disease or conditions associated with Alzheimer's disease, or autoimmune disease or conditions associated with autoimmune diseases. The subject is an animal subject, preferably a mammal, more preferably horses, cows, dogs, cats, sheep, pigs, mice, rats, monkeys, guinea pigs, and chickens, and most preferably a human.

According to the methods described herein, administration can be parenteral, pulmonary, oral, topical, intradermal, intramuscular, intraperitoneal, intravenous, subcutaneous, intranasal, epidural, ophthalmic, buccal, or rectal administration, as described below. In one embodiment, a composition described herein can be taken orally and prepared as one or more drops of the composition combined with a measure of water and imbibed by a subject.

An effective amount of a composition described herein is generally that which can reduce hyperproliferative cellular division so as to effect, for example, a reduction in tumor size or growth rate, or reduction of plasma glucose levels so as to effect, for example, a treatment for diabetes or pre-diabetes, a condition associated with diabetes or pre-diabetes, a treatment for an immune deficiency disease or disorder, including HIV, or conditions associated with an immune deficiency disease or disorder, a treatment for Alzheimer's disease or conditions associated with Alzheimer's disease, or a treatment for an autoimmune disease or conditions associated with autoimmune diseases. Other therapeutic effects include reduction or elimination of symptoms including, but not limited to, local symptoms, symptoms of metastasis, and systemic symptoms. Examples of local symptoms include, but are not limited to, unusual lumps or swelling, hemorrhage, pain or ulceration, and jaundice. Examples of symptoms of metastasis include, but are not limited to, enlarged lymph nodes, cough and hemoptysis, hepatomegaly, bone pain, fracture of affected bones, neurological symptoms, and pain. Examples of systemic symptoms include, but are not limited to, weight loss, poor appetite, fatigue and cachexia, excessive sweating (e.g., night sweats), anemia, and specific paraneoplastic phenomena (e.g., thrombosis or hormonal changes). In some embodiments, a composition is non-toxic to non-cancerous cells or beta cells and can result in minimal, if any, side effects to the subject.

The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by the attending physician within the scope of sound medical judgment.

When used in the methods described herein, a therapeutically effective amount of a composition described herein may be employed in pure form or, where such forms exist, in pharmaceutically acceptable salt form and with or without a pharmaceutically acceptable excipient. For example, compositions can be administered, at a reasonable benefit/risk ratio applicable to any medical treatment, in a sufficient amount to induce apoptosis in cells exhibiting hyperproliferative division, such as cancer cells, or in a sufficient amount to reduce serum blood glucose levels in subjects having diabetes, pre-diabetes, a condition associated with diabetes or pre-diabetes, a sufficient amount to treat or prevent an immune deficiency disease or disorder, including HIV, or conditions associated with immune deficiency disease or disorder, a sufficient amount to treat or prevent Alzheimer's disease or conditions associated with Alzheimer's disease, or a sufficient amount to treat or prevent an autoimmune disease or conditions associated with autoimmune diseases.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

Administration of a composition described herein can occur as a single event or over a time course of treatment. For example, compositions can be administered daily, weekly, bi-weekly, or monthly. The time course of treatment will usually be at least several days. Treatment can extend from several days to several weeks. For example, treatment could extend over one week, two weeks, or three weeks. Furthermore, treatment could extend from several weeks to several months or even a year or more. For example, for compositions used as a passive vaccination, a subject may continue to receive the composition repeatedly, like a booster. In some embodiments, compositions used as an active vaccination may result in lifetime immunity for the subject.

Treatment in accord with the methods described herein can be performed prior to, concurrent with, or after conventional treatment modalities, such as treatment with other diabetes or pre-diabetes, or immune deficiency disease or disorder, including HIV, drugs, drugs for Alzheimer's disease or conditions associated with Alzheimer's disease, or drugs for an autoimmune disease or conditions associated with autoimmune diseases, surgery, chemotherapy, radiation therapy, immunotherapy, or monoclonal antibody therapy. Preferably, treatment methods described herein occur prior to conventional treatment modalities, such as chemotherapy, so as to avoid debilitation of the subject's immune system and adverse effects on non-malignant cells. In some embodiments, administration of compositions described herein can ameliorate, reduce or eliminate damage to cells, tissues, or organs mediated by a chemotherapeutic agent or anti-cancer therapy. In some embodiments, administration of compositions described herein are is performed in a subject in which no (or substantially no) lymph tissue, lymph nodes, lymphatic channels, and/or lymph fluid is removed from the subject.

Assessment of relative success of the therapeutic treatment method can be monitored by the same or similar diagnostic approaches above including, but not limited to, blood tests, X-rays, CT scans, endoscopy or biopsy. Treatment in accord with the methods described herein can result in arrest or reduction of tumor growth, arrest or reduction in swelling surrounding a tumor, or reduction of blood glucose levels. For example, the therapeutic treatment method can reduce tumor growth rate, tumor size, or blood glucose levels by about 10%, about 20%, about 30%, about 40%, about 50%, about 60%, about 70%, about 80%, about 90%, about 95%, about 99%, or more. Preferably, the therapeutic treatment method completely eliminates the tumor, tumor growth rate, or returns blood glucose levels to normal.

### Administration

Compositions described herein can be administered in a variety of means known to the art. The agents can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration. In one embodiment, a composition described herein can be taken orally and prepared as one or more drops of the composition combined with a measure of water and imbibed by a subject. In some configurations, the oral formulation of the composition is tasteless.

Compositions comprising an agent described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents will be known to the skilled artisan and are within the scope of the invention.

Delivery systems may include, for example, an infusion pump which may be used to administer the agent in a manner similar to that used for delivering insulin or chemotherapy to specific organs or tumors. Typically, using such a system, the agent(s) is administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for biomolecular agent delivery can: provide for intracellular delivery; tailor biomolecule/agent release rates; increase the proportion of biomolecule that reaches its site of action; improve the transport of the drug to its site of action; allow co-localized deposition with other agents or excipients; improve the stability of the agent in vivo; prolong the residence time of the agent at its site of action by reducing clearance; decrease the nonspecific delivery of the agent to non-target tissues; decrease irritation caused by the agent; decrease toxicity due to high initial doses of the agent; alter the immunogenicity of the agent; decrease dosage frequency, improve taste of the product; or improve shelf life of the product.

### Kit

Another aspect of the invention is directed toward kits for the treatment of cancer, diabetes, pre-diabetes, conditions associated with diabetes or pre-diabetes, immune deficiency disease or disorder, including HIV, or conditions associated with immune deficiency disease or disorder, Alzheimer's disease or conditions associated with Alzheimer's disease, or an autoimmune disease or conditions associated with autoimmune diseases. Such kits can include the compositions of the present invention and, in certain embodiments, instructions for administration. Such kits can also contain diagnostic reagents for the detection of a biomarker in a biological sample. Various embodiments of the kit can facilitate performance of the methods described herein, for example, treatment methodologies. When supplied as a kit, the different components of the composition can be packaged in separate containers and admixed immediately before use. Components include, but are not limited to compositions or agents described herein, additional agents, genes encoding such agents, vectors, diagnostic reagents, assay reagents, or combinations thereof. Such packaging of the components separately can, if desired, be presented in a pack or dispenser device which may contain one or more unit dosage forms containing the composition(s). The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain lyophilized agent(s) and in a separate ampule, sterile water or sterile saline, each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention described herein.

### Example 1

### Preparation of L-S Product (“Protein B”)

L-S product was isolated via the following technique.

Actinomyces bovis (Hartz) (strain No. 10048, American Type Culture Collection, 12301 Parklawn Drive, Rockville, Md. U.S.A., 20852) was cultured in the following medium: 20 g/L N/Z-Case casein (Sheffield Chemical Company, Norwich, N.Y.); 10 g/L yeast Extract (Difco); 5 g/L NaCl; 0.006 g/L Fe SO₄; 0.077 g/L MgS0₄.7H₂O; 2 g/L glucose; 1 g/L agar; 10 g/L glycerol. The medium was brought to pH 7.6 with phosphate buffer before sterilization. The glucose was sterilized separately in a 50% solution (6 liters per 9 liter bottle for ½ hour at 250 D.) and added aseptically following sterilization. The production medium was dispensed in 9 liter bottles in 6 liter amounts. These containers were cooled rapidly after sterilization to insure anaerobiosis and the medium was immediately inoculated with A. bovis.

A horse was injected 4 to 8 times over one month with an non-infectious dead extract of the mold, Actinomyces bovis, to increase the concentration of anti-tumor, anti-diabetes or pre-diabetes, or anti-immune deficiency disease or disorder, including HIV, substance in the blood (first stage product). After one month, the horse was sacrificed, blood was collected, sodium citrate or heparin was added to the collected blood to prevent coagulation, and blood components were separated out by centrifugation to obtain a plasma sample.

A concentrated ether extract of citrated or heparinized plasma was made. Ether is then removed from the extract, providing ether soluble materials from horse citrated or heparinized plasma (second stage product). The ether soluble materials are then solubilized in benzene to provide ether-benzene soluble materials from horse citrated or heparinized plasma (third stage product). The ether-benzene soluble materials are then solubilized in water to provide ether-benzene-water soluble materials from horse plasma, yielding “crude” L-S product (fourth stage product).

The “crude” L-S product is extracted again with redistilled anhydrous ether, then redistilled benzene, and then anhydrous redistilled methanol. This is done primarily to remove those materials which are “trapped” by the fat during the ether and benzene extractions and which are soluble in water. The extracts are combined. The aqueous extract can be filtered through Berkfeldt filters of decreasing porosity until a clear filtrate is obtained. The filtrate is then evaporated to form a tan or yellowish powder. Components of the extract may include galacturonic acid, galactose, glucosamine, glucose, arabinose, xylose, palmitic acid, oleic acid, palmitoleic acid, myristic acid, stearic acid, G-15, G-17, lauric acid, and shorter chain acids (than creatine).

The powder can then be placed in a light mineral oil or sesame oil, stored in an ampoule, and sterilized in an autoclave for one hour at 270° C. Alternatively, it may be dissolved in physiological saline, but such solution is not stable for storage. Alternatively, it can be evaporated to produce residue. All solutions are kept refrigerated except the mineral oil solution. Residue is kept in a desiccator.

Alternatively the protein B can be prepared using an Ammonium Sulfate Fractionation Technique, which could reduce the time to express the anti-cancer serum from months to weeks, including expressing the material from the horse and checking the effectiveness of the material via a test in a mouse with natural breast cancer. General techniques for ammonium precipitation are well-known in the art (see, e.g., Wingfield, Curr. Protoc. Protein Sci. 2001, Appendix 3.

### Example 2

### Preparation of Protein B (PB-LPN) from Aspergillus Agent

An anti-cancer, anti-diabetes or anti-pre-diabetes, or immune deficiency disease or disorder, including HIV, therapeutic agent can be isolated from the serum of an animal pre-inoculated with Aspergillus and subsequently inoculated with Actinomyces bovis. Methods can be according to Example 1 but with Aspergillus as a pre-inoculation agent administered 10 days prior to Actinomyces bovis, which can decrease adverse reactions in the horse as compared to inoculation with Actinomyces bovis. One such composition formed in accord with such methods is PB-LPN. The treated horse was a full- or part-blooded Arabian horse. Serum is tested against titer at about 5 weeks. Blood is drawn from the horse and fractionated (e.g., into leukocytes, serum, and red blood cells). Serum is spun from blood to isolate immunoglobulin E (IgE) separate from other globulins, such as immunoglobulin G (IgG). Gamma globulins are reserved for possible use. The fractionation process and solid pellet isolation takes about 24 hours. The isolated fraction comprising IgE is freeze-dried for storage or stabilization, and then reconstituted with saline for administration.

The resulting composition containing an immune-stimulating antibody can be injected into a Bittner mouse, an animal model of cancer, to test efficacy, or another accepted animal cancer model. Alternatively, the resulting composition can be injected into an animal diabetes or pre-diabetes model. Alternatively, the resulting composition can be injected into an animal. Alternatively, the resulting composition can be injected into an animal immune deficiency disease or disorder, including HIV, model. The drug can be administered to human patients via oral administration or intramuscular injection.

A sample of the PB-LPN composition isolated from a horse injected with an Aspergillus inoculation agent is maintained in private deposit, is available to the USPTO upon request, and can be transferred to an International Depositary Authority prior to issue of the patent.

Alternatively the protein B can be prepared using an Ammonium Sulfate Fractionation Technique, which could reduce the time to express the anti-cancer serum from months to weeks, including expressing the material from the horse and checking the effectiveness of the material via a test in a mouse with natural breast cancer.

### Example 3

### Animal Studies with PB-LPN

Animal studies were conducted by injecting PB-LPN into a cancer animal model. Compositions are as described in Example 2, except as noted herein.

The results demonstrated a 40% eradication of breast tumors, a measurable degree of tumor reduction in 30%, and the remaining 30% showing little or no change, for an overall 70% elimination or reduction of solid tumor(s).

A subsequent study of a small number of test subjects, had a significantly greater tumor disappearance: 100% of breast cancer in 80% of mice without toxicity, and varying degrees of remission in the remaining 20%.

### Example 4

### Treatment of Walker 256 Rats with PB-LPN

In separate studies, 10,000 Walker 256 cells were injected subcutaneously on the backs of rats and subsequently inoculated with PB-LPN, glycerine alone, or control into the femoral vein. Results are provided in the tables below.

TABLE 1
Average tumor weight (grams) of survivors at 30 days and number of subjects
alive at day 40

| | “Treatment” given at time of | “Treatment” given at 17 days |
|---|---|---|
| | inoculation of cells | after cells |
| Treatment group | (number of subjects alive | (number of subjects alive |
| (10 rats each) | after 40 days) | after 40 days) |
| PB-LPN treated | 8.8 g (6) | 13.2 g (6) |
| Glycerine alone | 35.3 g (3) | 61.5 g (4) |
| Controls | 45.4 g (4) | 52.6 g (3) |

TABLE 2
Effect of PB-LPN (1000 mg/kg) on “takes” of the Walker 256 tumor

| | | | Average Day of | |
|---|---|---|---|---|
| | | | Appearance of | |
| Group | Number of Rats | “Takes” | Tumors | % Takes |
| Cells + PB-LPN | 20 | 8 | 23 | 40% |
| Cells + control | 10 | 10 | 7 | 100% |

TABLE 3
PB-LPN treatment of tumors (Walker 256 carcinoma)

| Group | Take/Day** | Day 1 | Day 10 | Day 20 | Day 30 | Day 40 |
|---|---|---|---|---|---|---|
| PB-LPN | 0 | 0 | 2 | 6 | 8 | 13*** |
| Treatment | | | | | | 4 deceased |
| (20 rats) | | | | | | |
| Saline | 0 | 0 | 4 | 10 | 10 | 10 deceased |
| Control | | | | | | |
| (10 rats) | | | | | | |
| Untreated | 0 | 0 | 5 | 9 | 10 | 10 deceased |
| (10 rats) | | | | | | |

**Figures represent cumulative tumors
***Number with total/partial reduction or disappearance of tumors
Two animals showed neither diminution or increase of size for a take of 16 alive in the period of the experiment.

### Example 5

### Treatment of Bittner'S Mouse Tumors with PB-LPN

The treatment of Bittner's mouse tumors was evaluated. Briefly, PB-LPN, glycerine/saline, saline, or normal protein was injected into a Bittner mouse with naturally occurring breast cancer to check its efficacy. Results from the study are presented below. Number of “takes” indicates the amount developing tumors at time of experiment. All mice had tumors at the time of initial treatment.

TABLE 4
Treatment of Bittner's mouse tumors (spontaneous)

| | | 30 Days | 35 Days | 40 Days |
|---|---|---|---|---|
| Number of Mice | Treatment | # Takes | # Second Control | # Final Alive |
| 20 ‘Z’ tumor in | PB-LPN | 6 | 5 | 14 |
| mice | | | | |
| 20 ‘A” tumor in | PB-LPN | 3 | 3 | 19 |
| mice | | | | |
| 20 A* mice | PB-LPN | 9 | 5 | 12 |
| 20 A mice | Glycerin/saline | 20 | 19 dead | 20 dead |
| 20 AZ strain | Saline | | | 20 deceased at |
| | | | | day 40 |
| 20 AZ strain | Normal protein | | | 20 deceased at |
| | | | | day 40 |

*mice which carried the MTA 6-8 months increased

### Example 6

### Treatment of Humans with PB-LPN

A clinical study of 35 cancer patients expressing lesions in one or more areas of the breast, skin, liver, lung, spine, pancreas, prostate, ovary, and stomach was conducted. Prior to the study, patient treatments ranged from no previous treatment (11 patients), to surgery, or surgery followed by chemotherapy. None of these prior treatments resulted in a decrease of pain suffered by the patients, or reduction of tumor(s) or metastases.

TABLE 5
Summary of 35 patients aggregately expressing cancer of the following
organs or sites independently, or as a result of metastases

| ORGAN | INCIDENCE |
|---|---|
| Adrenal Cortex | 1 |
| Bone | 3 |
| Brain | 1 |
| Breast | 5 |
| Chest | 1 |
| Colon | 2 |
| Glioma of the ocular nerve | 1 |
| Heart | 1 |
| Kidney | 2 |
| Leukemia | 3 |
| Liver | 6 |
| Lung | 1 |
| Lymph Nodes | 1 |
| Melanoma | 2 |
| Ovary | 1 |
| Pancreas | 3 |
| Prostate | 1 |
| Rectum | 1 |
| Spine | 1 |
| Spleen | 3 |
| Stomach | 6 |
| Testicle | 1 |
| Uterus | 1 |

Briefly, patients were administered PB-LPN via intramuscular injection.

Results of the study showed positive effects on several malignant sites without toxicity. In addition, some effectiveness was seen in an instance of lymphoid leukemia, and one of a glioma of the ocular nerve.

It was determined that the medication crosses the brain barrier. Such determination is based upon the observation that an embodiment of the composition described herein had a positive effect on the regression of a glioma tumor.

### Example 7

### Composition of Glass Ampule (GA) and Plastic Bottle (PB)

The following Examples are studies of protein B compositions that were used in patient treatment studies. The active materials in both samples (dubbed GA, for glass ampule, and PB for plastic bottle) were derived from a biological material which resulted from a pre-treated serum injected into a horse (a female Arabian—or part Arabian). Both antigenic agents injected into the horse comprise fungi. Both samples comprise Protein B.

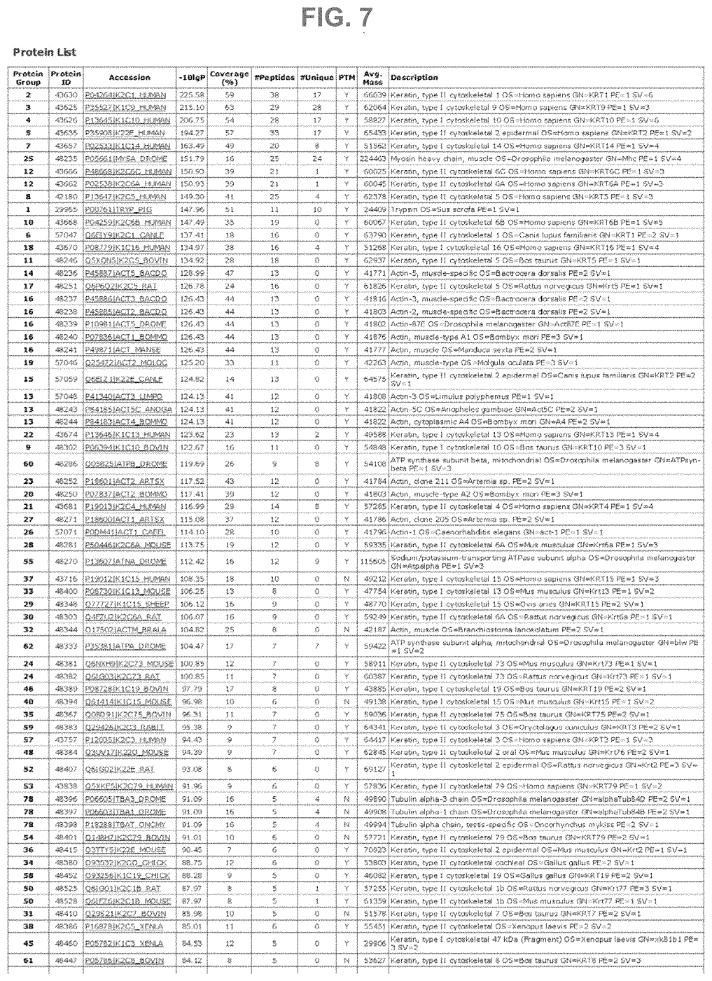

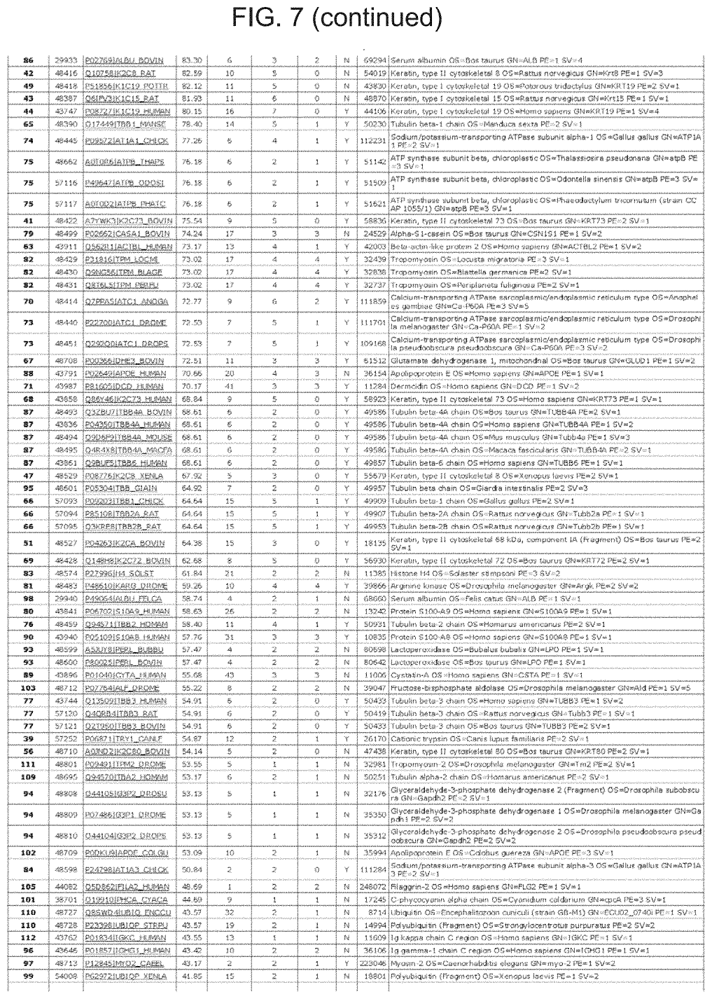

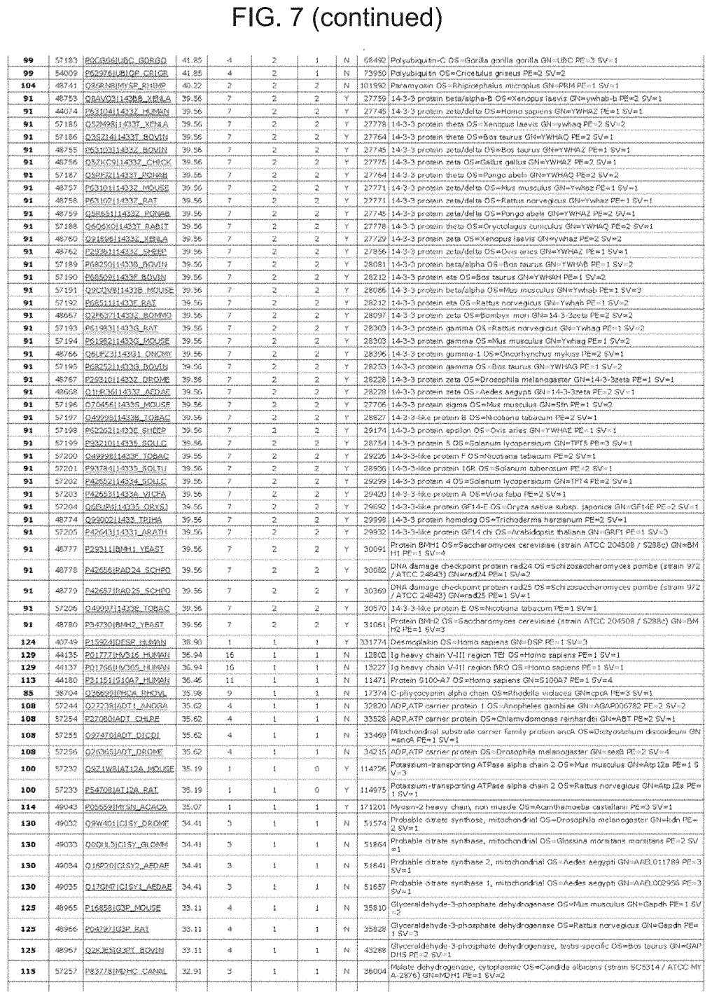

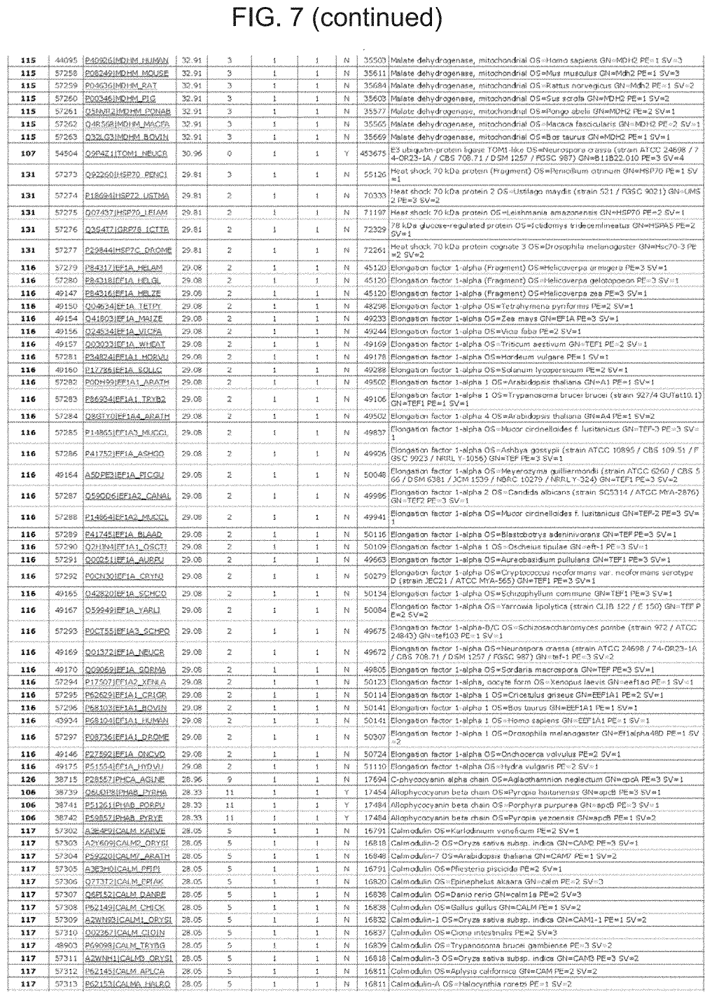

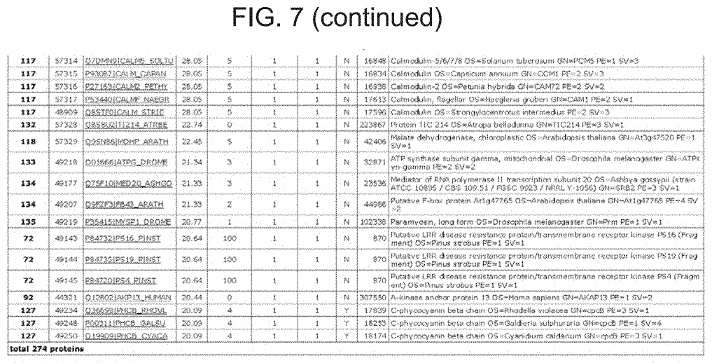

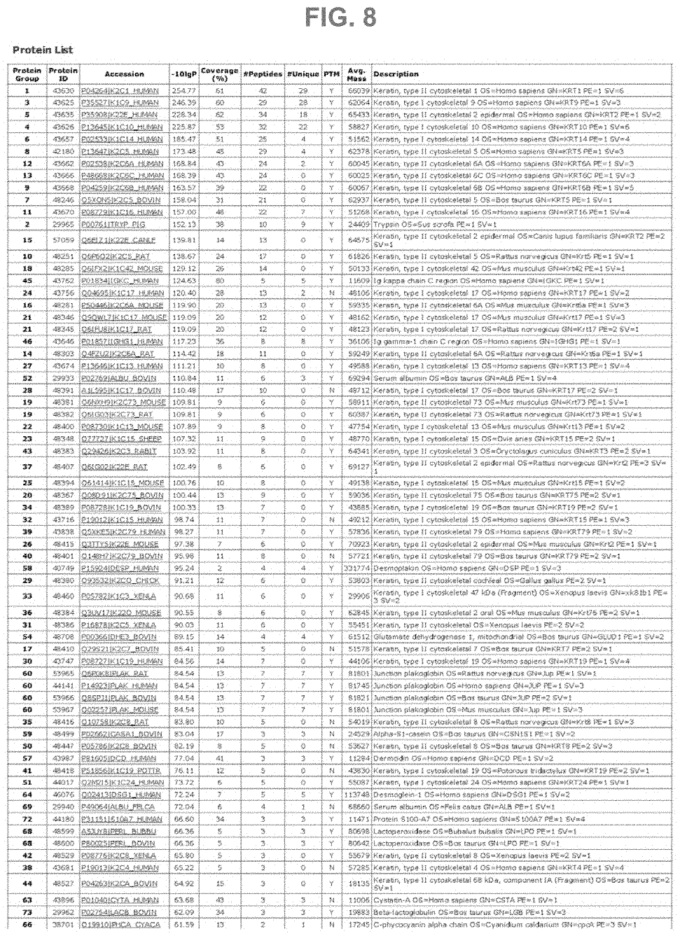

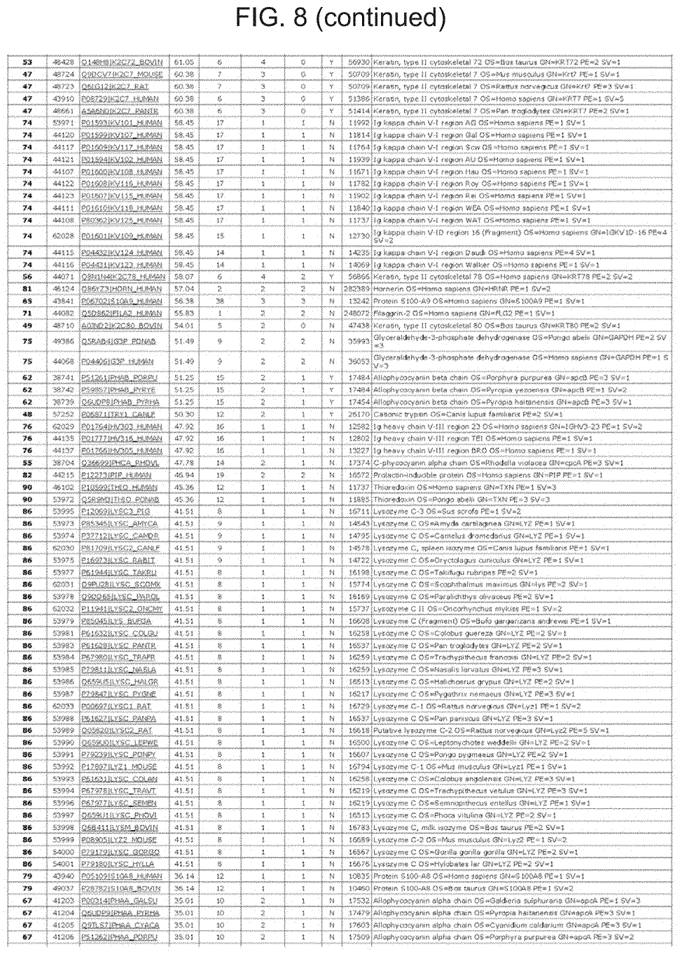

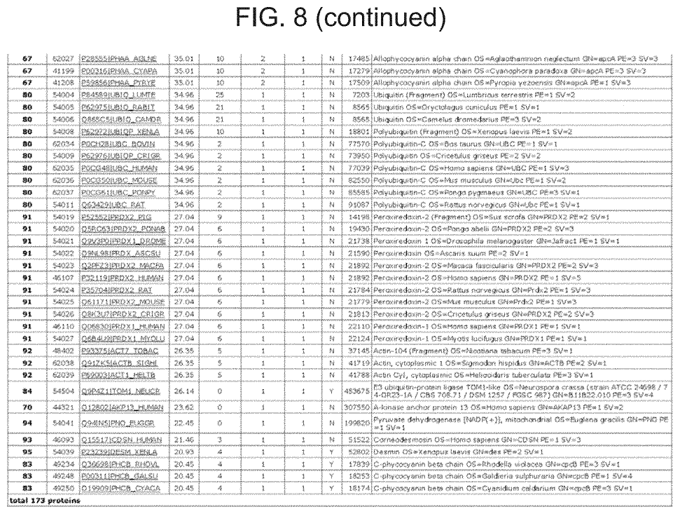

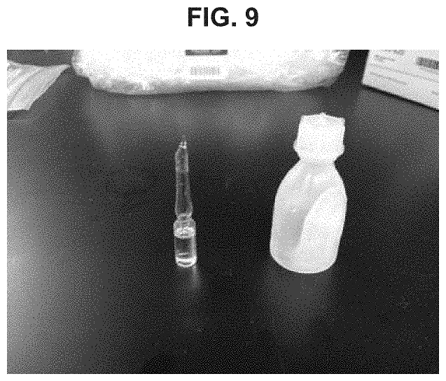

Alternatively a modified sample containing all or portion of the proteins listed in FIG. 7 and/or FIG. 8 may be used for treating patients.

The GA sample contains Aspergillus (thought to prevent anaphylactic shock in the animal) and A. bovis. The GA sample was the third generation of the material.

The plastic bottle (PB) sample was derived from the use of Actinomyces bovis, but not Aspergillus in the injection material. The PB sample was also used in oral treatments.

Both types of samples have been shown to show positive results (e.g., tumor regression) on tumors in mice, but has not yet been successful on transplanted tumors in mice.

### Example 8

### Composition Characteristics

It was found that the empirical crystalline formula of the compositions first incarnation was: C₂₅H₆₇N₁₈O₁₉. Its molecular weight was: 923.93. The compound was shown to fluoresce in ultra violet light, and has a small molecular size. The composition was found to be highly potent in doses of micrograms on cancers.

The composition was found to comprise: Galacturonic acid; Glucosamine; Glucose; Arabinose; Palmitic acid; Oleic acid; Palmitoleic acid; Myristic acid; Stearic acid; G-15; G-17; Lauric acid; and Shorter chain acids. Lauric acid is believed to treat viral infections and that cancer has a viral component. It is believed that the globulin contains the protein B anti-cancer/anti-diabetes/anti-pre-diabetes/anti-, immune deficiency disease or disorder, including HIV, agent.

### Example 9

### Characterization of Composition

The following example describes the characterization of the components in the Protein B compositions (in GA and PB sample containers).

### Samples

Plastic bottle with frozen liquid, similar to water ice. Sample designated now as “PB”.

Glass ampoule with slightly viscous, colored liquid. Sample designated now as “GA”.

Samples were thawed and aliquoted. Samples from the plastic bottle were labeled “PB” and samples from the glass ampoule were labeled “GA”. There was ˜5 mL of PB and 1.3 mL of GA. All aliquots but one of each sample were frozen at −20 C. Residue in glass ampoule rinsed with acetonitrile and used for HR ESI-MS. 200 μL of sample in plastic bottle also designated for HR ESI-MS.

4 μl from one aliquot each of PB and GA were diluted with 396 μl of acetonitrile for direct analysis by HR GC-MS (Daryl Giblin). In addition, 1 μl of each sample was derivatized with 100 μl of BSTFA/ACN (Sigma-Aldrich).

### GC MS Data

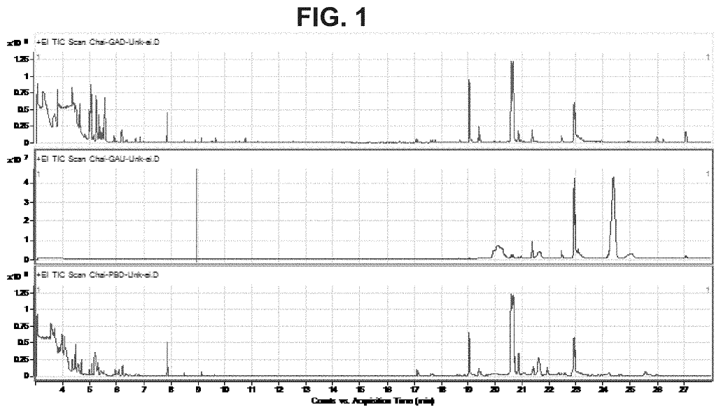

Samples were analyzed by GC-MS and the two samples were run both derivatized and underivatized. The results from the derivatized GA and PB vials are similar, though not identical. The early eluting peaks in FIG. 1, before 10 minutes are highly volatile small molecules. As such, peaks that elute after 19 minutes were of focus (see, e.g., Table 6). The high resolution Agilent 7200 GCQTOF acquired spectra during the entire scan, thus there is data gathered on the millisecond time scale. Every peak has a spectrum.

TABLE 6
List of compounds identified from abundant peaks

| Approx. time | Sample | Identification |
|---|---|---|
| 19.4 | | cis-7,10,13,16-Docosatetraenoic acid, |
| | | trimethylsilyl ester |
| 20.6 | | 9,12-Octadecadienoic acid (Z,Z)-, trimethylsilyl |
| | | ester |
| 20.7 | | trans-9-Octadecenoic acid, trimethylsilyl |
| | | ester |
| 20.9 | | Octadecanoic acid, trimethylsilyl ester |
| 21.4 | | another fatty ** |
| | | acid |
| 21.6 | PBD | Tocopherol |
| 22.6 | PBD | Eicosanoic acid, trimethylsilyl |
| | | ester |
| 22.9 | | Methyl 2-hydroxy-octadeca-9,12,15- |
| | | trienoate |
| 24.2 | PBD | Stigmastan-3,5-diene |
| 24.4 | GAD | β-Sitosterol trimethylsilyl ether |
| 25.6 | PBD | (+)-α-Tocopherol, O- |
| | | trimethylsilyl- |
| 26 | GAD | 1 -Monolinoleoylglycerol trimethylsilyl |
| | | ether |
| 26.02 | GAD | 1-Monooleoylglycerol trimethylsilyl ether |
| 27.1 | GAD | Squalene |

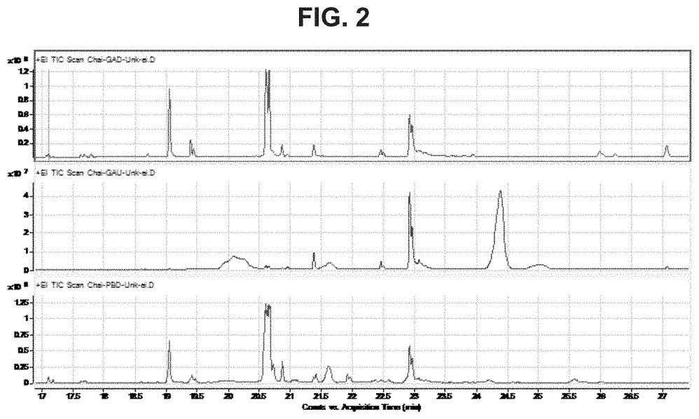

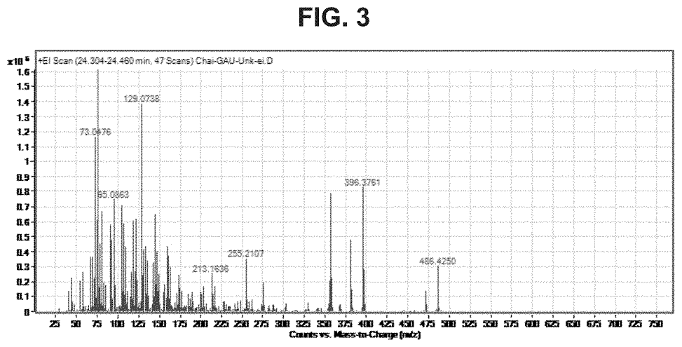

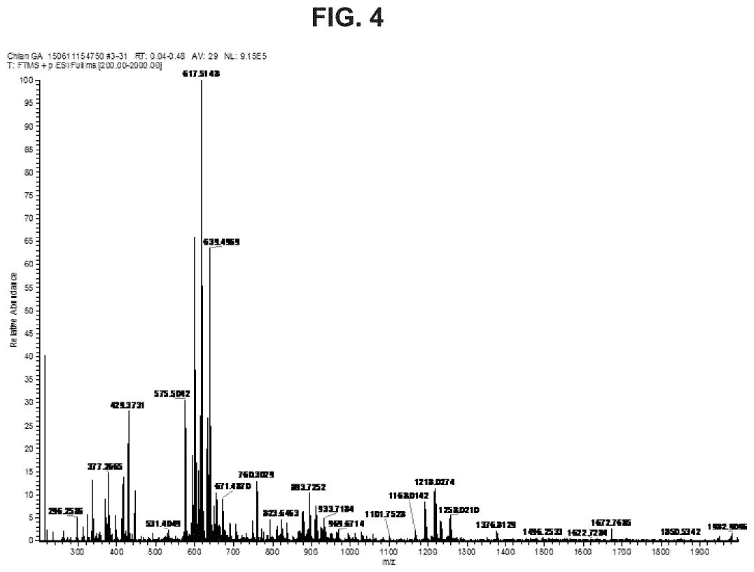

The table shows that there are several fatty acids and their derivatives in both mixtures. There are also some steroid derivatives. Note that the scales in FIG. 1 are different and most, but not all of the compounds appear in both samples. There are a lot of small peaks that did not get positive identification when searched against the NIST database, but they have similar fragmentation patterns and are thought to belong to the same classes of molecules. There was evidence that there are several kinds of derivatives (e.g., amidation), but no positive identification. FIG. 2 zooms in on the later elution times. FIG. 3 shows a typical mass spectrum with a confident identification.

### ESI-MS Data

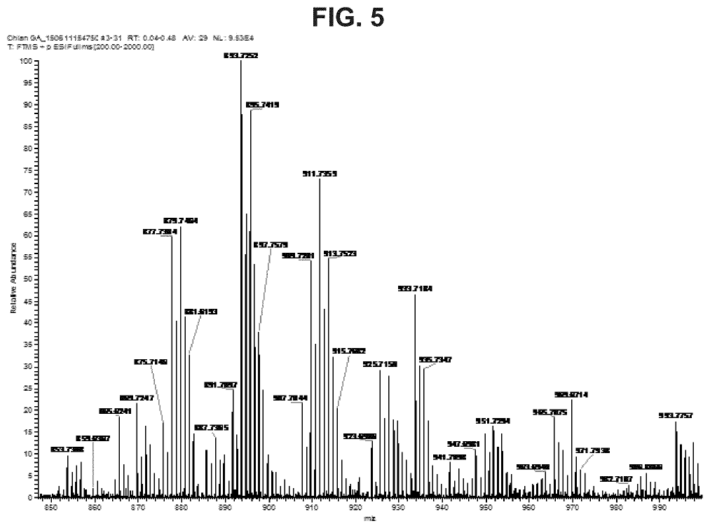

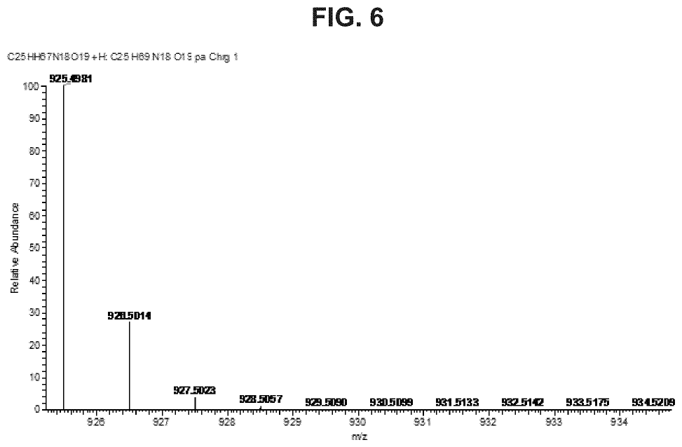

The samples were also analyzed using a Thermo LTQ-FT Ultra with an IonMax ESI source. FIG. 4 is an ESI-MS spectrum from GA sample. Evidence of peptides or proteins, were not observed. Everything in the data for both the PB and GA sample was singly charged. These are likely the lipids seen in the GC-MS experiments since there were many. No peak was found at 923.93 (FIG. 5). The predicted spectrum is shown in FIG. 6. Note that G-15 in the list of compounds from the correspondence is likely (3aS*,4R*,9bR*)-4-(6-Bromo-1,3-benzodioxol-5-yl)-3a,4,5,9b-3H-cyclopenta[c]quinolone (Tocris.com). G-17 was not found in a search of the literature, but it is thought there could be an analogous compound that is now identified by a different name.

### Summary

The GCMS data clearly shows a lot of fatty acids and sterols, and their derivatives. Future experiments include sifting through the smaller peaks and identifying them. The lack of a protein or peptide signal in ESI MS data could be due to the suppression of these signals by the lipids. Future experiments are aimed at processing the current aliquots further by extracting these lipids and then performing an LC-MS experiment on the remaining material.

### Example 10

### Sypro Gel Test

A Sypro gel test showed many proteins in the GA and PB samples.

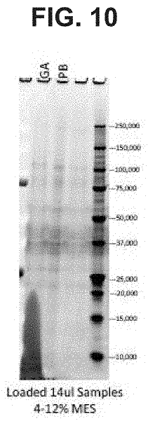

FIG. 10 shows an image from the gel used to analyze the two samples (GA=glass ampoule, PB=plastic bottle). The faint horizontal bands correspond to proteins. The data suggests that there are many proteins in the samples sample. The right lane is a molecular weight marker.

### Example 11

### In Vitro Organ-on-a-Chip Testing

The IgE and IgG fractions from a horse innoculated with PB-LPN are tested for their effect on various cancers by performing organ-on-a-chip testing. Examples of such organ-on-a-chip compositions are found in U.S. Pat. No. 11,059,041, incorporated herein by reference in its entirety. Briefly, various cancer tissues are placed in a multicellular, layered, microfluidic culture, and the effect of the IgE and IgG fractions from the immunized horse sera described herein on interactions between lamina propria-derived cells and epithelial cells and endothelial cells are studied. Organ-on-a-Chip technology is available commercially (EmulateBio Inc, Boston, MA), and permits the recreation of various human tumor microenvironments in vitro, allowing for mechanistic studies of cancer cell behavior and drug efficacy and safety, such as the efficacy of the immunized horse sera described herein. Endothelial co-culture provides important cell-cell interactions, while media flow and tissue-relevant stretch help model the mechanical forces cancer cells experience in the body. With these models, it is possible to modulate various cellular, molecular, chemical, and biophysical properties in a controlled manner to investigate their impact on cancer progression and behavior upon contact with the immunized horse sera described herein.

These microfluidic platforms or “chips” for testing and understanding cancer are used to understand the factors that contribute to cancer invading tissues and causing metastases. Tumor cells are grown on microfluidic devices with other non-cancerous tissues, including but not limited to, lamina propria-derived cells, stromal cells, epithelial cells, cells of the immune system, etc., under conditions that simulate tumor invasion. The interaction with the IgE and IgG fractions from the immunized horse sera described herein is tested to inhibit this activity.

The IgE and IgG fractions from a horse innoculated with PB-LPN are tested for their effect on diabetes by performing organ-on-a-chip testing. Conditions associated with diabetes or pre-diabetes refer generally to any clinical condition characterized by, or otherwise involving, an increased level of plasma glucose relative to a normal reference rate. Briefly, pancreatic tissue that generates insulin is placed in a multicellular, layered, microfluidic culture, and the effect of the IgE and IgG fractions from the immunized horse sera described herein on the ability of the diabetes compromised pancreatic cells to produce insulin are studied.

The IgE and IgG fractions from a horse innoculated with PB-LPN are tested for their effect on immune deficiency diseases, such as HIV, by performing organ-on-a-chip testing. Conditions associated with immune deficiency diseases or disorders refer generally to any clinical condition characterized by, or otherwise involving, a decreased level of immune cells or antibodies relative to a normal reference rate. Briefly, compromised immune cells are placed in a multicellular, layered, microfluidic culture, and the effect of the IgE and IgG fractions from the immunized horse sera described herein on the ability of the compromised immune cells to produce antibodies are studied.

The IgE and IgG fractions from a horse innoculated with PB-LPN are tested for their effect on Alzheimers by performing organ-on-a-chip testing. Conditions associated with Alzheimer's disease refer generally to any clinical condition characterized by, or otherwise involving, an increased level of β-amyloid protein or peptides relative to a normal reference rate. Briefly, brain tissue that generates β-amyloid protein or peptides is placed in a multicellular, layered, microfluidic culture, and the effect of the IgE and IgG fractions from the immunized horse sera described herein on the ability of the brain cells to decrease the production of β-amyloid protein or peptides are studied.

The IgE and IgG fractions from a horse innoculated with PB-LPN are tested for their effect on autoimmune disease by performing organ-on-a-chip testing. Conditions associated with autoimmune diseases refer generally to any clinical condition characterized by, or otherwise involving, an increased level of autoantibodies relative to a normal reference rate. Briefly, tissue that generates autoantibodies are placed in a multicellular, layered, microfluidic culture, and the effect of the IgE and IgG fractions from the immunized horse sera described herein on the ability of the tissue to decrease or cease the production of autoantibodies are studied.

### Incorporation by Reference

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A composition for the treatment of conditions associated with hyperproliferative cellular division, diabetes, pre-diabetes, conditions associated with diabetes or pre-diabetes, immune deficiency disease or disorder, or conditions associated with immune deficiency disease or disorder, Alzheimer's disease or conditions associated with Alzheimer's disease, or an autoimmune disease or conditions associated with autoimmune diseases, comprising:
an extract of a biological sample of an animal inoculated with an antigenic agent comprising an Ascomyceta;
wherein the extract exhibits cytotoxic activity towards cells exhibiting conditions associated with hyperproliferative cellular division, or enhancement of beta cells in diabetes, pre-diabetes, conditions associated with diabetes or pre-diabetes, immune deficiency disease or disorder, or conditions associated with immune deficiency disease or disorder, Alzheimer's disease or conditions associated with Alzheimer's disease, or an autoimmune disease or conditions associated with autoimmune diseases.

2. The composition of claim 1 further comprising a pharmaceutically acceptable carrier or excipient.

3. A method of producing a composition for treatment of conditions associated with hyperproliferative cellular division, diabetes, pre-diabetes, conditions associated with diabetes or pre-diabetes, immune deficiency disease or disorder, or conditions associated with immune deficiency disease or disorder, Alzheimer's disease or conditions associated with Alzheimer's disease, or an autoimmune disease or conditions associated with autoimmune diseases, comprising:
inoculating an animal with an antigenic agent comprising an Ascomyceta;
obtaining a biological sample from the inoculated animal;
extracting the biological sample with an organic solvent to produce an aqueous phase and an organic phase; and
isolating the organic phase;
wherein the organic phase comprises an anti-cancer composition that exhibits cytotoxic activity towards cells exhibiting conditions associated with hyperproliferative cellular division, or enhancement of beta cells in diabetes, pre-diabetes, conditions associated with diabetes or pre-diabetes, immune deficiency disease or disorder, or conditions associated with immune deficiency disease or disorder, Alzheimer's disease or conditions associated with Alzheimer's disease, or an autoimmune disease or conditions associated with autoimmune diseases.

4. The method of claim 3 wherein the organic solvent comprises one or more solvents selected from the group consisting of hydrocarbons, halogenated hydrocarbons, alcohol, ketone, ester, and ether.

5. The method of claim 4 wherein the solvent hydrocarbon is selected from at least one of
the group consisting of n-hexane, n-heptane, petroleum ether, and cyclohexane, or
the group consisting of chloroform, dichloromethane and carbon tetrachloride.

6. The method of claim 4 wherein the solvent alcohol is selected from the group consisting of methanol, ethanol, propanol and butanol.

7. The method of claim 4 wherein the solvent ester is at least one of ethyl acetate or diethyl ether.

8. The method of claim 3 further comprising at least two extraction steps.

9. The method of claim 8 further comprising at least three extraction steps.

10. The method of claim 8 comprising a first extraction with ether, a second extraction with benzene, and a third extraction with water.

11. A method of treating a condition associated with hyperproliferative cellular division, diabetes, pre-diabetes, a condition associated with diabetes or pre-diabetes, immune deficiency disease or disorder, or condition associated with immune deficiency disease or disorder, Alzheimer's disease or conditions associated with Alzheimer's disease, or an autoimmune disease or conditions associated with autoimmune diseases, in a subject comprising administering a therapeutically effective amount of a composition of claim 1 to a subject in need thereof.

12. A method of treating a condition associated with hyperproliferative cellular division, diabetes, pre-diabetes, a condition associated with diabetes or pre-diabetes, immune deficiency disease or disorder, or condition associated with immune deficiency disease or disorder, Alzheimer's disease or conditions associated with Alzheimer's disease, or an autoimmune disease or conditions associated with autoimmune diseases, in a subject comprising administering a therapeutically effective amount of a composition obtained from the method of claim 3 to a subject in need thereof.

13. The method of claim 11, wherein the subject is diagnosed with cancer, diagnosed as at risk for cancer, diagnosed with diabetes or pre-diabetes, diagnosed as at risk for diabetes or pre-diabetes, immune deficiency disease or disorder, or condition associated with immune deficiency disease or disorder, Alzheimer's disease or conditions associated with Alzheimer's disease, or an autoimmune disease or conditions associated with autoimmune diseases.

14. The method of claim 11, wherein the condition associated with hyperproliferative cellular division is selected from the group consisting of a solid cancer and a hematopoietic malignant cancer.

15. The method of claim 11, wherein the condition associated with hyperproliferative cellular division is selected from the group consisting of gastrointestinal tumors, cancer of liver and biliary tract, pancreatic cancer, prostatic cancer, testicular cancer, lung cancer, breast cancer, malignant melanoma, ovarian cancer, uterine cancer, cervical cancer, cancer of the head and neck, bladder cancer, sarcomas and osteosarcomas, Kaposi sarcoma, AIDS-related Kaposi sarcoma, and renal carcinoma.

16. The method of claim 11 wherein administration is parenteral, pulmonary, oral, topical, intradermal, intramuscular, intraperitoneal, intravenous, subcutaneous, intranasal, epidural, ophthalmic, buccal, or rectal administration.

17. The method of claim 11, wherein the condition associated with diabetes or pre-diabetes is cardiovascular disease.

18. The composition of claim 1 wherein the actinic agent comprises an Aspergillus spp.

19. The composition of claim 18 wherein the Aspergillus spp. is selected from the group consisting of Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus israelii, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowi, Aspergillus tamari, Aspergillus terreus, Aspergillus ustus, and Aspergillus versicolor.

20. The composition of claim 1, wherein the actinic agent further comprises a virus, bacteria, protozoa, or fungi.

21. The composition of claim 20 wherein the actinic agent further comprises an Actinomyces spp.

22. The composition of claim 21 wherein the Actinomyces spp. is Actinomyces bovis.

23. The composition of claim 1 wherein the actinic agent is a macerated actinic agent.

24. The composition of claim 1 wherein the composition is substantially non-toxic to non-cancerous cells or beta cells.

25. The composition of claim 1 wherein the biological sample comprises one or more of whole blood, plasma, serum, pleural fluid, cerebrospinal fluid, or culture fluid.

26. The composition of claim 25, wherein the biological sample comprises one or more of plasma or serum.

27. The composition of claim 1 wherein the biological sample comprises an immune stimulating antibody.

28. The composition of claim 1 wherein the biological sample comprises:
cis-7,10,13,16-Docosatetraenoic acid, trimethylsilyl ester;
9,12-Octadecadienoic acid (Z,Z)-, trimethylsilyl ester;
trans-9-Octadecenoic acid, trimethylsilyl ester;
octadecanoic acid, trimethylsilyl ester;
a fatty acid;
tocopherol;
eicosanoic acid, trimethylsilyl ester;
methyl 2-hydroxy-octadeca-9,12,15-trienoate;
stigmastan-3,5-diene;
β-sitosterol trimethylsilyl ether;
(+)-α-tocopherol, O-trimethylsilyl-1-monolinoleoylglycerol trimethylsilyl ether;
1-monooleoylglycerol trimethylsilyl ether;
squalene,
(3aS*,4R*,9bR*)-4-(6-Bromo-1,3-benzodioxol-5-yl)-3a,4,5,9b-3H-cyclopenta[c]quinolone;
a sterol;
galacturonic acid;
glucosamine;
glucose;
arabinose;
palmitic acid;
oleic acid;
palmitoleic acid;
myristic acid;
stearic acid;
G-15;
G-17;
lauric acid; or
derivatives thereof, optionally, amidation or acetylation derivatives thereof.

29. A kit for treatment of a condition associated with hyperproliferative cellular division, diabetes, pre-diabetes, a condition associated with diabetes or pre-diabetes, immune deficiency disease or disorder, or condition associated with immune deficiency disease or disorder, Alzheimer's disease or conditions associated with Alzheimer's disease, or an autoimmune disease or conditions associated with autoimmune diseases, comprising a composition of claim 1.

30. The composition of claim 1, wherein the immune deficiency disease or disorder is HIV.

31. A device, comprising:
a microfluidic platform comprising;
a tissue associated with a predetermined medical condition, wherein the tissue is configured in a multicellular, layered arrangement;
a composition as recited in claim 1;
a passage connecting the composition and the tissue; and
a pump controlling a movement of the composition toward the tissue.

32. The device of claim 31, further comprising a detector, wherein the detector detects an interaction between the composition and the tissue.

33. The device of claim 32, further comprising a processor, wherein the processor analyzes the interaction between the composition and the tissue.

34. The device of claim 33, wherein the processor determines whether the composition affects the predetermined medical condition.

35. The device of claim 34, wherein the effect of the composition is at least one of mitigation of the medical condition, reduction of the severity of the medical condition, increasing the activity of the tissue effected by the medical condition, or reducing the activity of the tissue effected by the medical condition.

36. The device of claim 31, wherein the medical condition is at least one of cancer, diabetes, pre-diabetes, conditions associated with diabetes or pre-diabetes, immune deficiency diseases and disorders, HIV, conditions associated with immune deficiency diseases and disorders, Alzheimer's disease and conditions associated with Alzheimer's disease, and autoimmune disease and conditions associated with autoimmune diseases.
